SEQUENCE LISTING (21 sequences)

SEQ ID NO: 1 - protein, 925 aa, Homo sapiens
MERDGCAGGGSRGGEGGRAPREGPAGNGRDRGRSHAAEAPGDPQAAASLLAPMDVGEEPLEKAARARTAKDPNTYKVLSLVLSVCVLTTILGCIFGLKPSCAKEVKSCKGRCFERTFGNCRCDAACVELGNCCLDYQETCIEPEHIWTCNKFRCGEKRLTRSLCACSDDCKDKGDCCINYSSVCQGEKSWVEEPCESINEPQCPAGFETPPTLLFSLDGFRAEYLHTWGGLLPVISKLKKCGTYTKNMRPVYPTKTFPNHYSIVTGLYPESHGIIDNKMYDPKMNASFSLKSKEKFNPEWYKGEPIWVTAKYQGLKSGTFFWPGSDVEINGIFPDIYKMYNGSVPFEERILAVLQWLQLPKDERPHFYTLYLEEPDSSGHSYGPVSSEVIKALQRVDGMVGMLMDGLKELNLHRCLNLILISDHGMEQGSCKKYIYLNKYLGDVKNIKVIYGPAARLRPSDVPDKYYSFNYEGIARNLSCREPNQHFKPYLKHFLPKRLHFAKSDRIEPLTFYLDPQWQLALNPSERKYCGSGFHGSDNVFSNMQALFVGYGPGFKHGIEADTFENIEVYNLMCDLLNLTPAPNNGTHGSLNHLLKNPVYTPKHPKEVHPLVQCPFTRNPRDNLGCSCNPSILPIEDFQTQFNLTVAEEKIIKHETLPYGRPRVLQKENTICLLSQHQFMSGYSQDILMPLWTSYTVDRNDSFSTEDFSNCLYQDFRIPLSPVHKCSFYKNNTKVSYGFLSPPQLNKNSSGIYSEALLTTNIVPMYQSFQVIWRYFHDTLLRKYAEERNGVNVVSGPVFDFDYDGRCDSLENLRQKRRVIRNQEILIPTHFFIVLTSCKDTSQTPLHCENLDTLAFILPHRTDNSESCVHGKHDSSWVEELLMLHRARITDVEHITGLSFYQQRKEPVSDILKLKTHLPTFSQED

SEQ ID NO: 2 - protein, 827 aa, Homo sapiens
PSCAKEVKSCKGRCFERTFGNCRCDAACVELGNCCLDYQETCIEPEHIWTCNKFRCGEKRLTRSLCACSDDCKDKGDCCINYSSVCQGEKSWVEEPCESINEPQCPAGFETPPTLLFSLDGFRAEYLHTWGGLLPVISKLKKCGTYTKNMRPVYPTKTFPNHYSIVTGLYPESHGIIDNKMYDPKMNASFSLKSKEKFNPEWYKGEPIWVTAKYQGLKSGTFFWPGSDVEINGIFPDIYKMYNGSVPFEERILAVLQWLQLPKDERPHFYTLYLEEPDSSGHSYGPVSSEVIKALQRVDGMVGMLMDGLKELNLHRCLNLILISDHGMEQGSCKKYIYLNKYLGDVKNIKVIYGPAARLRPSDVPDKYYSFNYEGIARNLSCREPNQHFKPYLKHFLPKRLHFAKSDRIEPLTFYLDPQWQLALNPSERKYCGSGFHGSDNVFSNMQALFVGYGPGFKHGIEADTFENIEVYNLMCDLLNLTPAPNNGTHGSLNHLLKNPVYTPKHPKEVHPLVQCPFTRNPRDNLGCSCNPSILPIEDFQTQFNLTVAEEKIIKHETLPYGRPRVLQKENTICLLSQHQFMSGYSQDILMPLWTSYTVDRNDSFSTEDFSNCLYQDFRIPLSPVHKCSFYKNNTKVSYGFLSPPQLNKNSSGIYSEALLTTNIVPMYQSFQVIWRYFHDTLLRKYAEERNGVNVVSGPVFDFDYDGRCDSLENLRQKRRVIRNQEILIPTHFFIVLTSCKDTSQTPLHCENLDTLAFILPHRTDNSESCVHGKHDSSWVEELLMLHRARITDVEHITGLSFYQQRKEPVSDILKLKTHLPTFSQED

SEQ ID NO: 3 - protein, 1058 aa, synthetic construct
  REGION 1..1058: note = source = /note="Description of Artificial Sequence: Synthetic polypeptide"
PSCAKEVKSCKGRCFERTFGNCRCDAACVELGNCCLDYQETCIEPEHIWTCNKFRCGEKRLTRSLCACSDDCKDKGDCCINYSSVCQGEKSWVEEPCESINEPQCPAGFETPPTLLFSLDGFRAEYLHTWGGLLPVISKLKKCGTYTKNMRPVYPTKTFPNHYSIVTGLYPESHGIIDNKMYDPKMNASFSLKSKEKFNPEWYKGEPIWVTAKYQGLKSGTFFWPGSDVEINGIFPDIYKMYNGSVPFEERILAVLQWLQLPKDERPHFYTLYLEEPDSSGHSYGPVSSEVIKALQRVDGMVGMLMDGLKELNLHRCLNLILISDHGMEQGSCKKYIYLNKYLGDVKNIKVIYGPAARLRPSDVPDKYYSFNYEGIARNLSCREPNQHFKPYLKHFLPKRLHFAKSDRIEPLTFYLDPQWQLALNPSERKYCGSGFHGSDNVFSNMQALFVGYGPGFKHGIEADTFENIEVYNLMCDLLNLTPAPNNGTHGSLNHLLKNPVYTPKHPKEVHPLVQCPFTRNPRDNLGCSCNPSILPIEDFQTQFNLTVAEEKIIKHETLPYGRPRVLQKENTICLLSQHQFMSGYSQDILMPLWTSYTVDRNDSFSTEDFSNCLYQDFRIPLSPVHKCSFYKNNTKVSYGFLSPPQLNKNSSGIYSEALLTTNIVPMYQSFQVIWRYFHDTLLRKYAEERNGVNVVSGPVFDFDYDGRCDSLENLRQKRRVIRNQEILIPTHFFIVLTSCKDTSQTPLHCENLDTLAFILPHRTDNSESCVHGKHDSSWVEELLMLHRARITDVEHITGLSFYQQRKEPVSDILKLKTHLPTFSQEDPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 1068 aa, synthetic construct
  REGION 1..1068: note = source = /note="Description of Artificial Sequence: Synthetic polypeptide"
PSCAKEVKSCKGRCFERTFGNCRCDAACVELGNCCLDYQETCIEPEHIWTCNKFRCGEKRLTRSLCACSDDCKDKGDCCINYSSVCQGEKSWVEEPCESINEPQCPAGFETPPTLLFSLDGFRAEYLHTWGGLLPVISKLKKCGTYTKNMRPVYPTKTFPNHYSIVTGLYPESHGIIDNKMYDPKMNASFSLKSKEKFNPEWYKGEPIWVTAKYQGLKSGTFFWPGSDVEINGIFPDIYKMYNGSVPFEERILAVLQWLQLPKDERPHFYTLYLEEPDSSGHSYGPVSSEVIKALQRVDGMVGMLMDGLKELNLHRCLNLILISDHGMEQGSCKKYIYLNKYLGDVKNIKVIYGPAARLRPSDVPDKYYSFNYEGIARNLSCREPNQHFKPYLKHFLPKRLHFAKSDRIEPLTFYLDPQWQLALNPSERKYCGSGFHGSDNVFSNMQALFVGYGPGFKHGIEADTFENIEVYNLMCDLLNLTPAPNNGTHGSLNHLLKNPVYTPKHPKEVHPLVQCPFTRNPRDNLGCSCNPSILPIEDFQTQFNLTVAEEKIIKHETLPYGRPRVLQKENTICLLSQHQFMSGYSQDILMPLWTSYTVDRNDSFSTEDFSNCLYQDFRIPLSPVHKCSFYKNNTKVSYGFLSPPQLNKNSSGIYSEALLTTNIVPMYQSFQVIWRYFHDTLLRKYAEERNGVNVVSGPVFDFDYDGRCDSLENLRQKRRVIRNQEILIPTHFFIVLTSCKDTSQTPLHCENLDTLAFILPHRTDNSESCVHGKHDSSWVEELLMLHRARITDVEHITGLSFYQQRKEPVSDILKLKTHLPTFSQEDPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKDDDDDDDDDD

SEQ ID NO: 5 - protein, 819 aa, Homo sapiens
SCKGRCFERTFGNCRCDAACVELGNCCLDYQETCIEPEHIWTCNKFRCGEKRLTRSLCACSDDCKDKGDCCINYSSVCQGEKSWVEEPCESINEPQCPAGFETPPTLLFSLDGFRAEYLHTWGGLLPVISKLKKCGTYTKNMRPVYPTKTFPNHYSIVTGLYPESHGIIDNKMYDPKMNASFSLKSKEKFNPEWYKGEPIWVTAKYQGLKSGTFFWPGSDVEINGIFPDIYKMYNGSVPFEERILAVLQWLQLPKDERPHFYTLYLEEPDSSGHSYGPVSSEVIKALQRVDGMVGMLMDGLKELNLHRCLNLILISDHGMEQGSCKKYIYLNKYLGDVKNIKVIYGPAARLRPSDVPDKYYSFNYEGIARNLSCREPNQHFKPYLKHFLPKRLHFAKSDRIEPLTFYLDPQWQLALNPSERKYCGSGFHGSDNVFSNMQALFVGYGPGFKHGIEADTFENIEVYNLMCDLLNLTPAPNNGTHGSLNHLLKNPVYTPKHPKEVHPLVQCPFTRNPRDNLGCSCNPSILPIEDFQTQFNLTVAEEKIIKHETLPYGRPRVLQKENTICLLSQHQFMSGYSQDILMPLWTSYTVDRNDSFSTEDFSNCLYQDFRIPLSPVHKCSFYKNNTKVSYGFLSPPQLNKNSSGIYSEALLTTNIVPMYQSFQVIWRYFHDTLLRKYAEERNGVNVVSGPVFDFDYDGRCDSLENLRQKRRVIRNQEILIPTHFFIVLTSCKDTSQTPLHCENLDTLAFILPHRTDNSESCVHGKHDSSWVEELLMLHRARITDVEHITGLSFYQQRKEPVSDILKLKTHLPTFSQED

SEQ ID NO: 6 - protein, 739 aa, Homo sapiens
EKSWVEEPCESINEPQCPAGFETPPTLLFSLDGFRAEYLHTWGGLLPVISKLKKCGTYTKNMRPVYPTKTFPNHYSIVTGLYPESHGIIDNKMYDPKMNASFSLKSKEKFNPEWYKGEPIWVTAKYQGLKSGTFFWPGSDVEINGIFPDIYKMYNGSVPFEERILAVLQWLQLPKDERPHFYTLYLEEPDSSGHSYGPVSSEVIKALQRVDGMVGMLMDGLKELNLHRCLNLILISDHGMEQGSCKKYIYLNKYLGDVKNIKVIYGPAARLRPSDVPDKYYSFNYEGIARNLSCREPNQHFKPYLKHFLPKRLHFAKSDRIEPLTFYLDPQWQLALNPSERKYCGSGFHGSDNVFSNMQALFVGYGPGFKHGIEADTFENIEVYNLMCDLLNLTPAPNNGTHGSLNHLLKNPVYTPKHPKEVHPLVQCPFTRNPRDNLGCSCNPSILPIEDFQTQFNLTVAEEKIIKHETLPYGRPRVLQKENTICLLSQHQFMSGYSQDILMPLWTSYTVDRNDSFSTEDFSNCLYQDFRIPLSPVHKCSFYKNNTKVSYGFLSPPQLNKNSSGIYSEALLTTNIVPMYQSFQVIWRYFHDTLLRKYAEERNGVNVVSGPVFDFDYDGRCDSLENLRQKRRVIRNQEILIPTHFFIVLTSCKDTSQTPLHCENLDTLAFILPHRTDNSESCVHGKHDSSWVEELLMLHRARITDVEHITGLSFYQQRKEPVSDILKLKTHLPTFSQED

SEQ ID NO: 7 - protein, 232 aa, Homo sapiens
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 1051 aa, synthetic construct
  REGION 1..1051: note = source = /note="Description of Artificial Sequence: Synthetic polypeptide"
SCKGRCFERTFGNCRCDAACVELGNCCLDYQETCIEPEHIWTCNKFRCGEKRLTRSLCACSDDCKDKGDCCINYSSVCQGEKSWVEEPCESINEPQCPAGFETPPTLLFSLDGFRAEYLHTWGGLLPVISKLKKCGTYTKNMRPVYPTKTFPNHYSIVTGLYPESHGIIDNKMYDPKMNASFSLKSKEKFNPEWYKGEPIWVTAKYQGLKSGTFFWPGSDVEINGIFPDIYKMYNGSVPFEERILAVLQWLQLPKDERPHFYTLYLEEPDSSGHSYGPVSSEVIKALQRVDGMVGMLMDGLKELNLHRCLNLILISDHGMEQGSCKKYIYLNKYLGDVKNIKVIYGPAARLRPSDVPDKYYSFNYEGIARNLSCREPNQHFKPYLKHFLPKRLHFAKSDRIEPLTFYLDPQWQLALNPSERKYCGSGFHGSDNVFSNMQALFVGYGPGFKHGIEADTFENIEVYNLMCDLLNLTPAPNNGTHGSLNHLLKNPVYTPKHPKEVHPLVQCPFTRNPRDNLGCSCNPSILPIEDFQTQFNLTVAEEKIIKHETLPYGRPRVLQKENTICLLSQHQFMSGYSQDILMPLWTSYTVDRNDSFSTEDFSNCLYQDFRIPLSPVHKCSFYKNNTKVSYGFLSPPQLNKNSSGIYSEALLTTNIVPMYQSFQVIWRYFHDTLLRKYAEERNGVNVVSGPVFDFDYDGRCDSLENLRQKRRVIRNQEILIPTHFFIVLTSCKDTSQTPLHCENLDTLAFILPHRTDNSESCVHGKHDSSWVEELLMLHRARITDVEHITGLSFYQQRKEPVSDILKLKTHLPTFSQEDEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 1046 aa, synthetic construct
  REGION 1..1046: note = source = /note="Description of Artificial Sequence: Synthetic polypeptide"
SCKGRCFERTFGNCRCDAACVELGNCCLDYQETCIEPEHIWTCNKFRCGEKRLTRSLCACSDDCKDKGDCCINYSSVCQGEKSWVEEPCESINEPQCPAGFETPPTLLFSLDGFRAEYLHTWGGLLPVISKLKKCGTYTKNMRPVYPTKTFPNHYSIVTGLYPESHGIIDNKMYDPKMNASFSLKSKEKFNPEWYKGEPIWVTAKYQGLKSGTFFWPGSDVEINGIFPDIYKMYNGSVPFEERILAVLQWLQLPKDERPHFYTLYLEEPDSSGHSYGPVSSEVIKALQRVDGMVGMLMDGLKELNLHRCLNLILISDHGMEQGSCKKYIYLNKYLGDVKNIKVIYGPAARLRPSDVPDKYYSFNYEGIARNLSCREPNQHFKPYLKHFLPKRLHFAKSDRIEPLTFYLDPQWQLALNPSERKYCGSGFHGSDNVFSNMQALFVGYGPGFKHGIEADTFENIEVYNLMCDLLNLTPAPNNGTHGSLNHLLKNPVYTPKHPKEVHPLVQCPFTRNPRDNLGCSCNPSILPIEDFQTQFNLTVAEEKIIKHETLPYGRPRVLQKENTICLLSQHQFMSGYSQDILMPLWTSYTVDRNDSFSTEDFSNCLYQDFRIPLSPVHKCSFYKNNTKVSYGFLSPPQLNKNSSGIYSEALLTTNIVPMYQSFQVIWRYFHDTLLRKYAEERNGVNVVSGPVFDFDYDGRCDSLENLRQKRRVIRNQEILIPTHFFIVLTSCKDTSQTPLHCENLDTLAFILPHRTDNSESCVHGKHDSSWVEELLMLHRARITDVEHITGLSFYQQRKEPVSDILKLKTHLPTFSQEDDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 971 aa, synthetic construct
  REGION 1..971: note = source = /note="Description of Artificial Sequence: Synthetic polypeptide"
EKSWVEEPCESINEPQCPAGFETPPTLLFSLDGFRAEYLHTWGGLLPVISKLKKCGTYTKNMRPVYPTKTFPNHYSIVTGLYPESHGIIDNKMYDPKMNASFSLKSKEKFNPEWYKGEPIWVTAKYQGLKSGTFFWPGSDVEINGIFPDIYKMYNGSVPFEERILAVLQWLQLPKDERPHFYTLYLEEPDSSGHSYGPVSSEVIKALQRVDGMVGMLMDGLKELNLHRCLNLILISDHGMEQGSCKKYIYLNKYLGDVKNIKVIYGPAARLRPSDVPDKYYSFNYEGIARNLSCREPNQHFKPYLKHFLPKRLHFAKSDRIEPLTFYLDPQWQLALNPSERKYCGSGFHGSDNVFSNMQALFVGYGPGFKHGIEADTFENIEVYNLMCDLLNLTPAPNNGTHGSLNHLLKNPVYTPKHPKEVHPLVQCPFTRNPRDNLGCSCNPSILPIEDFQTQFNLTVAEEKIIKHETLPYGRPRVLQKENTICLLSQHQFMSGYSQDILMPLWTSYTVDRNDSFSTEDFSNCLYQDFRIPLSPVHKCSFYKNNTKVSYGFLSPPQLNKNSSGIYSEALLTTNIVPMYQSFQVIWRYFHDTLLRKYAEERNGVNVVSGPVFDFDYDGRCDSLENLRQKRRVIRNQEILIPTHFFIVLTSCKDTSQTPLHCENLDTLAFILPHRTDNSESCVHGKHDSSWVEELLMLHRARITDVEHITGLSFYQQRKEPVSDILKLKTHLPTFSQEDEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 966 aa, synthetic construct
  REGION 1..966: note = source = /note="Description of Artificial Sequence: Synthetic polypeptide"
EKSWVEEPCESINEPQCPAGFETPPTLLFSLDGFRAEYLHTWGGLLPVISKLKKCGTYTKNMRPVYPTKTFPNHYSIVTGLYPESHGIIDNKMYDPKMNASFSLKSKEKFNPEWYKGEPIWVTAKYQGLKSGTFFWPGSDVEINGIFPDIYKMYNGSVPFEERILAVLQWLQLPKDERPHFYTLYLEEPDSSGHSYGPVSSEVIKALQRVDGMVGMLMDGLKELNLHRCLNLILISDHGMEQGSCKKYIYLNKYLGDVKNIKVIYGPAARLRPSDVPDKYYSFNYEGIARNLSCREPNQHFKPYLKHFLPKRLHFAKSDRIEPLTFYLDPQWQLALNPSERKYCGSGFHGSDNVFSNMQALFVGYGPGFKHGIEADTFENIEVYNLMCDLLNLTPAPNNGTHGSLNHLLKNPVYTPKHPKEVHPLVQCPFTRNPRDNLGCSCNPSILPIEDFQTQFNLTVAEEKIIKHETLPYGRPRVLQKENTICLLSQHQFMSGYSQDILMPLWTSYTVDRNDSFSTEDFSNCLYQDFRIPLSPVHKCSFYKNNTKVSYGFLSPPQLNKNSSGIYSEALLTTNIVPMYQSFQVIWRYFHDTLLRKYAEERNGVNVVSGPVFDFDYDGRCDSLENLRQKRRVIRNQEILIPTHFFIVLTSCKDTSQTPLHCENLDTLAFILPHRTDNSESCVHGKHDSSWVEELLMLHRARITDVEHITGLSFYQQRKEPVSDILKLKTHLPTFSQEDDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 15 aa, Homo sapiens
EPKSCDKTHTCPPCP

SEQ ID NO: 14 - protein, 10 aa, Homo sapiens
DKTHTCPPCP

SEQ ID NO: 15 - protein, 14 aa, Homo sapiens
PKSCDKTHTCPPCP

SEQ ID NO: 16 - protein, 15 aa, synthetic construct
  REGION 1..15: note = source = /note="Description of Artificial Sequence: Synthetic peptide"
GGGGSGGGGSGGGGS

SEQ ID NO: 17 - protein, 6 aa, Homo sapiens
PSCAKE

SEQ ID NO: 18 - protein, 10 aa, synthetic construct
  REGION 1..10: note = source = /note="Description of Artificial Sequence: Synthetic peptide"
DDDDDDDDDD

SEQ ID NO: 19 - protein, 50 aa, synthetic construct
  REGION 1..50: note = MISC_FEATURE - /note="This sequence may encompass 1-10 'Gly Gly Gly Gly Ser' repeating units"
  REGION 1..50: note = source = /note="Description of Artificial Sequence: Synthetic polypeptide"
GGGGSGGGGSGGGGSGGGGSGGGGSGGGGSGGGGSGGGGSGGGGSGGGGS

SEQ ID NO: 20 - protein, 8 aa, synthetic construct
  REGION 1..8: note = source = /note="Description of Artificial Sequence: Synthetic peptide"
DDDDDDDD

SEQ ID NO: 21 - protein, 8 aa, synthetic construct
  REGION 1..8: note = source = /note="Description of Artificial Sequence: Synthetic peptide"
EEEEEEEE

(19) United States
(12) Patent Application Publication
(10) Pub. No.: US 2025/0019677 A1
(43) Pub. Date: Jan. 16, 2025

(54) METHODS OF IMPROVING CARDIOVASCULAR FUNCTION AND TREATING CARDIOVASCULAR DISEASE USING A RECOMBINANT ECTONUCLEOTIDE PYROPHOSPHATASE PHOSPHODIESTERASE (NPP1)

(71) Applicant: Inozyme Pharma, Inc., Boston, MA (US)

(72) Inventors: Tayeba Khan, Lexington, MA (US); Andre Marozsan, Killingworth, CT (US); Kim Lynette Askew, Lincoln, MA (US)

(21) Appl. No.: 18/904,634

(22) Filed: Oct. 2, 2024

Related U.S. Application Data
(63) Continuation of application No. 16/648,388, filed on Mar. 18, 2020, now Pat. No. 12,134,790.
(60) Provisional application No. 62/563,829, filed on Sep. 27, 2017.

(51) Int. Cl. C12N 9/16; A61K 9/00; A61K 45/06; A61P 9/10; C07K 19/00

(52) U.S. Cl. CPC C12N 9/16; A61P 9/10; C07K 19/00; A61K 9/0019; A61K 45/06; C12Y 301/04001

(57) ABSTRACT

The present invention provides methods for improving cardiovascular function in a human patient (e.g., reducing hypertension), as well as methods of treating a cardiovascular disease, by administering a recombinant human soluble ectonucleotide pyrophosphatase phosphodiesterase (hsNPP1), active fragment or fusion protein thereof.

### RELATED INFORMATION PARAGRAPH

This application is a continuation application of U.S. patent application Ser. No. 16/648,388, filed on Mar. 18, 2020, which is a 35 U.S.C. 371 national stage filing of International Application No. PCT/US2018/052795, filed on Sep. 26, 2018, which claims the benefit of the priority date of U.S. Provisional Application No. 62/563,829, filed on Sep. 27, 2017, the contents of each of which are hereby incorporated by reference in their entirety.

### SEQUENCE LISTING

This application contains a Sequence Listing which has been submitted electronically as a WIPO Standard ST.26 XML file via Patent Center, created on Oct. 2, 2024, is entitled “4427-12307.xml” and is 33.7 KB in size. The sequence listing is incorporated herein by reference in its entirety.

### BACKGROUND

The cardiovascular system, also known as the circulatory system, includes the heart, arteries, veins, capillaries and blood. The heart functions as the pump that moves blood through the body. The arterial circulation delivers blood from the heart to the body, and the venous circulation carries it back to the heart. Capillaries are tiny blood vessels at the interface of the arterial and venous circulation where exchange of substances between the blood and body tissues occurs. The three main functions of the cardiovascular system are (1) the transport of nutrients, oxygen, and hormones to cells throughout the body and removal of metabolic wastes (carbon dioxide, nitrogenous wastes), (2) protection of the body by white blood cells, antibodies, and complement proteins that circulate in the blood and defend the body against foreign microbes and toxins, as well as clotting mechanisms that protect the body from blood loss after injuries, and (3) regulation of body temperature, fluid pH, and water content of cells.

Cardiovascular diseases are the leading cause of death globally. Cardiovascular disease includes coronary artery diseases (CAD) (such as angina and myocardial infarction (commonly known as a heart attack), stroke, heart failure, hypertensive heart disease, rheumatic heart disease, cardiomyopathy, heart arrhythmia, congenital heart disease, valvular heart disease, carditis, aortic aneurysms, peripheral artery disease, thromboembolic disease, and venous thrombosis (see GBD 2013 Mortality and Causes of Death, Collaborators (17 Dec. 2014), Lancet. 385 (9963): 117-71).

Approximately 85 million people in the United States have hypertension (also known as “high blood pressure”). Normal blood pressure is 120 over 80 mm of mercury (mmHg). Medical guidelines define hypertension as a blood pressure higher than 140 over 90 millimeters of mercury (mmHg). Blood pressure is the force exerted by the blood against the walls of the blood vessels. The systolic reading of 140 mmHg refers to the pressure as the heart pumps blood around the body. The diastolic reading of 90 mmHg refers to the pressure as the heart relaxes and refills with blood. Hypertension severely impacts quality of life and increases the risk of heart disease, stroke, and death.

In spite of considerable research in the field, there is a continuing need for therapies to effectively treat cardiovascular diseases, including hypertension.

### SUMMARY OF THE INVENTION

The present invention relates to uses of isolated recombinant human soluble NPP1 that lacks N-terminal cytosolic and transmembrane domains and fusion proteins thereof for improving cardiovascular function in a human patient, including preventing and/or treating cardiovascular diseases, such as hypertension. Any cardiovascular disorder is within the scope of the present invention.

In one aspect, method for improving cardiovascular function in a human patient are provided, the method comprising administering to the patient one or more doses of a recombinant human soluble ectonucleotide pyrophosphatase phosphodiesterase (hsNPP1), active fragment or fusion protein thereof. In one embodiment, the improvement is a reduction in elevated blood pressure (e.g., reduction of blood pressure higher than 140 over 90 millimeters of mercury (mmHg) to within a normal blood pressure range (about 120 over 80 mm of mercury (mmHg)). In another embodiment, the improvement is a reduction in elevated blood pressure by at least 5 (e.g., at least 10, 15, 20, 25, 30, 35, 40, 45, 50, 55, 60, 65, or 70) %. In another embodiment, the improvement is a reduction in elevated blood pressure by about 1.5-fold, 2-fold, 2.5-fold, 3-fold, 3.5-fold, or 4-fold. In another embodiment, the improvement is a normalization of blood pressure (e.g., to about 120 over 80 mm of mercury (mmHg)). In another embodiment, the improvement is a reduction in left ventricular end-diastolic pressure (EDP) and end-systolic pressure (ESP) (e.g., by at least 5, 10, 15, 20, 25, 30, 35, 40, 45, 50, 55, 60, 65, or 70%). In another embodiment, the improvement is a reduction in left ventricular EDP and ESP by about 1.5-fold, 2-fold, 2.5-fold, 3-fold, 3.5-fold, or 4-fold. In another embodiment, the improvement is a reduction in ventricle stiffness (EDPVR) (e.g., by at least 5, 10, 15, 20, 25, 30, 35, 40, 45, 50, 55, 60, 65, or 70%). In another embodiment, the improvement is a reduction in ventricle stiffness by about 1.5-fold, 2-fold, 2.5-fold, 3-fold, 3.5-fold, or 4-fold. In another embodiment, the improvement is in increase in contractility (PRSW) (e.g., by at least 5, 10, 15, 20, 25, 30, 35, 40, 45, 50, 55, 60, 65, or 70%). In another embodiment, the improvement is in increase in contractility by about 1.5-fold, 2-fold, 2.5-fold, 3-fold, 3.5-fold, or 4-fold.

In another aspect, methods for treating a human patient having a cardiovascular disorder are provided, the method comprising administering to the patient one or more doses of a recombinant human soluble ectonucleotide pyrophosphatase phosphodiesterase (hsNPP1), active fragment or fusion protein thereof. Exemplary cardiovascular diseases include, but are not limited to, coronary artery diseases (CAD) (such as angina and myocardial infarction (commonly known as a heart attack), stroke, heart failure, hypertensive heart disease, rheumatic heart disease, cardiomyopathy, heart arrhythmia, congenital heart disease, valvular heart disease, carditis, aortic aneurysms, peripheral artery disease, thromboembolic disease, and venous thrombosis.

In another aspect, methods for treating a human patient having hypertension are provided, the method comprising administering to the patient one or more doses of a recombinant human soluble ectonucleotide pyrophosphatase phosphodiesterase (hsNPP1), active fragment or fusion protein thereof.

In another aspect, methods of treating a human patient having hypertension are provided, the method comprising: a) identifying a human patient as having hypertension and b) administering to the identified patient one or more doses of a recombinant human soluble ectonucleotide pyrophosphatase phosphodiesterase (hsNPP1), active fragment or fusion protein thereof.

In another aspect, methods for reducing hypertension in a human patient are provided, the method comprising administering to the patient one or more doses of a recombinant human soluble ectonucleotide pyrophosphatase phosphodiesterase (hsNPP1), active fragment or fusion protein thereof.

In one embodiment, the methods described herein result in a reduction in the patient's blood pressure (e.g., from an elevated blood pressure higher than about 140 over 90 millimeters of mercury (mmHg) to within a normal blood pressure range (about 120 over 80 mm of mercury (mmHg)). In another embodiment, the patient's blood pressure is reduced by at least 5 (e.g., at least 10, 15, 20, 25, 30, 35, 40, 45, 50, 55, 60, 65, or 70) % after treatment. In another embodiment, the patient's blood pressure is reduced by about 1.5-fold, 2-fold, 2.5-fold, 3-fold, 3.5-fold, or 4-fold after treatment. In another embodiment, the patient's blood pressure is normalized after treatment (e.g., to about 120 over 80 mm of mercury (mmHg)).

In one embodiment, a NPP1 fusion protein is administered. Preferred fusion proteins comprise and NPP1 component an Fc region of an immunoglobulin and, optionally, a targeting moiety. In one embodiment, the targeting moiety is Asp₁₀ (SEQ ID NO: 18). In another embodiment, the targeting moiety comprises at least eight consecutive aspartic acid or glutamic acid residues (SEQ ID NOS 20 and 21, respectively). Particular NPP1 fusion proteins for administration in accordance with the methods disclosed herein have the amino acid sequence set forth in SEQ ID NO:3, SEQ ID NO:4, SEQ ID NO:9, SEQ ID NO:10, SEQ ID NO:11 or SEQ ID NO:12.

Any suitable amount of the recombinant hsNPP1 can be administered to the human patient. In one embodiment, the hsNPP1 is administered in one or more doses containing about 0.5 mg/kg, 1.0 mg/kg, 2.0 mg/kg, 3.0 mg/kg, 4.0 mg/kg, 5.0 mg/kg, 6.0 mg/kg, 7.0 mg/kg, 8.0 mg/kg, 9.0 mg/kg, 10.0 mg/kg, 11.0 mg/kg, 12.0 mg/kg, 13.0 mg/kg, 14.0 mg/kg, 15.0 mg/kg, 16.0 mg/kg, 17.0 mg/kg, 18.0 mg/kg, 19.0 mg/kg, or 20.0 mg/kg. In another embodiment, the hsNPP1 is administered in one or more doses containing about 1.0 mg/kg to about 5.0 mg/kg NPP1. In another embodiment, the hsNPP1 is administered in one or more doses containing about 1.0 mg/kg to about 10.0 mg/kg NPP1.

The time period between doses of the hsNPP1 is at least 2 days and can be longer, for example at least 3 days, at least 1 week, 2 weeks or 1 month. In one embodiment, the administration is weekly, bi-weekly, or monthly.

The recombinant hsNPP1 can be administered in any suitable way, such as intravenously, subcutaneously, or intraperitoneally.

The recombinant hsNPP1 can be administered in combination with one or more additional therapeutic agents. Exemplary therapeutic agents include, but are not limited to a thiazide diuretic (e.g., hydrochlorothiazide (Microzide) or chlorthalidone), a beta blocker (e.g., acebutolol (Sectral) or atenolol (Tenormin)), an angiotensin-converting enzyme (ACE) inhibitor (e.g., lisinopril (Zestril), benazepril (Lotensin), or captopril (Capoten)), an angiotensin II receptor blocker (ARB) (e.g., candesartan (Atacand) or losartan (Cozaar)), a calcium channel blocker (e.g., amlodipine (Norvasc) or diltiazem (Cardizem)), a renin inhibitor (e.g., Aliskiren (Tekturna)), an alpha blocker (e.g., doxazosin (Cardura) or prazosin (Minipress)), an alpha-beta blocker (e.g., carvedilol (Coreg) or labetalol (Trandate)), a central-acting agent (e.g., clonidine (Catapres, Kapvay), guanfacine (Intuniv, Tenex) and methyldopa), a vasodilator (e.g., hydralazine and minoxidil), and/or an aldosterone antagonist (e.g., spironolactone (Aldactone) or eplerenone (Inspra)). In one embodiment, the recombinant hsNPP1 and additional therapeutic agent are administered separately and are administered concurrently or sequentially. In one embodiment, the recombinant hsNPP1 is administered prior to administration of the additional therapeutic agent. In another embodiment, the recombinant hsNPP1 is administered after administration of the additional therapeutic agent. In another embodiment, the recombinant hsNPP1 and additional therapeutic agent are administered together.

In another aspect uses of an isolated recombinant human sNPP1, fragment or fusion protein thereof are provided. In one embodiment, the use of an isolated recombinant human sNPP1, fragment or fusion protein thereof for the manufacture of a medicament for improving cardiovascular function is provided. In another embodiment, the use of an isolated recombinant human sNPP1, fragment or fusion protein thereof for the manufacture of a medicament for reducing hypertension is provided. In another embodiment, the invention provides the use of an isolated recombinant human sNPP1, fragment or fusion protein thereof for reducing hypertension. In another embodiment, the invention provides the use of an isolated recombinant human sNPP1, fragment or fusion protein thereof for treating hypertension. In another embodiment, the invention provides the use of an isolated recombinant human sNPP1, fragment or fusion protein thereof for improving cardiovascular function in a patient.

Other features and advantages of the invention will be apparent from the following detailed description and claims.

### BRIEF DESCRIPTION OF THE DRAWINGS

FIGS. 1A-1E show that there was no reduction in calcification (FIGS. 1A-1C), no increase in plasma pyrophosphate (PPi) levels (FIG. 1D), and high plasma anti-drug antibody (“ADA”) levels (FIG. 1E) in ENPP1-treated 2J mice after 6 weeks of treatment.

FIG. 2 shows that there was no increase in pyrophosphate (PPi) levels in ENPP1-treated 2J mice, twenty-four hours after the last dose of a six week treatment.

FIGS. 3A-3D show that Enpp1-treatment reduces elevated blood pressure in Asj-2J mice.

FIGS. 4A-4B show that Enpp1-treatment reduces elevated left ventricular end-diastolic pressures (EDP) and end-systolic (ESP) pressures in Asj-2J mice.

FIGS. 5A-5C is data from pressure volume loops that indicates that Enpp1-treatment reduces ventricle stiffness (EDPVR) and increases contractility (PRSW) in Asj-2J mice.

FIGS. 6A-6J is echocardiogram data which suggests that Asj-2J mice do not have left ventricular hypertrophy (no change in LV Diastolic Anterior/Posterior Wall Thickness, internal dimensions, End-Diastolic Area, and Estimated LV Mass).

### DETAILED DESCRIPTION

### Definitions

Unless defined otherwise, all technical and scientific terms used herein have the same meaning as commonly understood by one of ordinary skill in the art to which this invention belongs. Although any methods and materials similar or equivalent to those described herein can be used in practice or testing of the present invention, the preferred methods and materials are described.

For clarity, “NPP1” and “ENPP1” refer to the same protein and are used interchangeably herein.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

As used herein, the term “normal,” when used to modify the term “individual” or “subject” refers to an individual or group of individuals who does/do not have a particular disease or condition (e.g., a cardiovascular disorder) and is also not suspected of having or being at risk for developing the disease or condition. The term “normal” is also used herein to qualify a biological specimen or sample isolated from a normal or healthy individual or subject (or group of such subjects), for example, a “normal control sample” or “normal control”.

As used herein, the term “fragment”, with regard to NPP1 proteins, refers to an active subsequence of the full-length NPP1. A “fragment” of a protein or peptide can be at least about 20 amino acids in length; for example, at least about 50 amino acids in length; at least about 100 amino acids in length; at least about 200 amino acids in length; at least about 300 amino acids in length; or at least about 400 amino acids in length (and any integer value in between). The fragments may range in size from four amino acid residues to the entire amino acid sequence minus one amino acid. Thus, a protein “comprising at least a portion of the amino acid sequence of SEQ ID NO: 1” encompasses the full-length NPP1 and fragments thereof.

An “isolated” or “purified” soluble NPP1 protein or biologically active fragment or fusion protein thereof is substantially free of cellular material or other contaminating proteins from the cell or tissue source from which the NPP1 protein, biologically active fragment or NPP1 fusion protein is derived, or substantially free from chemical precursors or other chemicals when chemically synthesized. The language “substantially free of cellular material” includes preparations of NPP1 protein, biologically active fragment, or NPP1 fusion protein in which the protein is separated from cellular components of the cells from which it is isolated or recombinantly produced. In one embodiment, the language “substantially free of cellular material” includes preparations of NPP1 protein, biologically active fragment or NPP1 fusion protein having less than about 30% (by dry weight) of non-NPP1 protein/fragment/fusion protein (also referred to herein as a “contaminating protein”), more preferably less than about 20% of non-NPP1 protein/fragment/fusion protein, still more preferably less than about 10% of non-NPP1 protein/fragment/fusion protein, and most preferably less than about 5% non-NPP1 protein/fragment/fusion protein. When the NPP1 protein, fusion protein, or biologically active fragment thereof is recombinantly produced, it is also preferably substantially free of culture medium, i.e., culture medium represents less than about 20%, more preferably less than about 10%, and most preferably less than about 5% of the volume of the protein preparation.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

As used herein, the term “subject” encompasses mammals and non-mammals. Examples of mammals include, but are not limited to, humans, chimpanzees, apes monkeys, cattle, horses, sheep, goats, swine, rabbits, dogs, cats, rats, mice, guinea pigs, and the like. Examples of non-mammals include, but are not limited to, birds, fish and the like.

As used herein, the term “therapeutically effective amount” refers to a nontoxic but sufficient amount of an agent (e.g., hsNPP1 proteins) which, as compared to a corresponding subject who has not received such amount, results in improved treatment, healing, prevention, or amelioration of a disease, disorder, or side effect, or a decrease in the rate of advancement of a disease or disorder (e.g., a cardiac disease or disorder). The term also includes within its scope amounts effective to enhance normal physiological function.

As used herein, a cardiovascular disease or disorder is one that involves the heart or blood vessels. Exemplary cardiovascular diseases include, but are not limited to, coronary artery diseases (CAD) (such as angina and myocardial infarction (commonly known as a heart attack), stroke, heart failure, hypertension, rheumatic heart disease, cardiomyopathy, heart arrhythmia, congenital heart disease, valvular heart disease, carditis, aortic aneurysms, peripheral artery disease, thromboembolic disease, and venous thrombosis.

As used herein, “hypertension” (also known as “high blood pressure”) is defined as a blood pressure higher than 140 over 90 millimeters of mercury (mmHg). Blood pressure is the force exerted by the blood against the walls of the blood vessels. The systolic reading of 140 mmHg refers to the pressure as the heart pumps blood around the body. The diastolic reading of 90 mmHg refers to the pressure as the heart relaxes and refills with blood. Normal blood pressure is 120 over 80 mm of mercury (mmHg). Hypertension severely impacts quality of life and increases the risk of heart disease, stroke, and death.

High blood pressure that is not caused by another condition or disease is called “primary” or “essential” hypertension. If the hypertension occurs as a result of another condition, it is called “secondary” hypertension. Primary hypertension can result from multiple factors, including blood plasma volume, activity of the hormones that regulate of blood volume and pressure, and environmental factors, such as stress and lack of exercise. Secondary hypertension has specific causes and is a complication of another problem (e.g., diabetes, due to both kidney problems and nerve damage, kidney disease, pheochromocytoma (a rare cancer of an adrenal gland), Cushing syndrome (which can be caused by corticosteroid drugs), congenital adrenal hyperplasia (disorder of the cortisol-secreting adrenal glands), hyperthyroidism (overactive thyroid gland), hyperparathyroidism (which affects calcium and phosphorous levels), pregnancy, sleep apnea, obesity, and/or chronic kidney disease (CKD).

As used herein “heart rate” (HR) refers to the speed of the heartbeat measured by the number of contractions of the heart per minute (bpm). The heart rate can vary according to the body's physical needs, including the need to absorb oxygen and excrete carbon dioxide. It is usually equal or close to the pulse measured at any peripheral point. Activities that can provoke change include physical exercise, sleep, anxiety, stress, illness, and ingestion of drugs. Many sources cite the normal resting adult human heart rate as ranging from 60-100 bpm (see, e.g., “Target Heart Rates”. American Heart Association. 4 Apr. 2014). Tachycardia is a fast heart rate, defined as above 100 bpm at rest (see, e.g., “Tachycardia, Fast Heart Rate”. American Heart Association. 2 May 2013). Bradycardia is a slow heart rate, defined as below 60 bpm at rest. Several studies, as well as expert consensus indicates that the normal resting adult heart rate is probably closer to a range between 50 and 90 bpm (see, e.g., Aladin, et al., The American Journal of Cardiology. 114 (11): 1701-06 (2014-12-01); Hjalmarson, A., et al., The American Journal of Cardiology. 65 (9): 547-53 (1990-03-01); Spodick, D. H., The American Journal of Cardiology. 72 (5): 487-88 (1993-08-15); and Mason, Jay W. et al., Journal of Electrocardiology. 40 (3): 228-34 (2007-07-01).

As used herein, “fractional shortening” (FS) is the reduction of the length of the end-diastolic diameter, or fraction of any diastolic dimension, that occurs by the end of or is lost in the systole.

As used herein, “arterial blood pressure” in the larger vessels consists of several distinct components: systolic and diastolic pressures, pulse pressure, and mean arterial pressure.

When “systemic arterial blood pressure” (SAP) is measured, it is recorded as a ratio of two numbers (e.g., 120/80 is a normal adult blood pressure), expressed as systolic pressure over diastolic pressure. The systolic pressure is the higher value (typically around 120 mm Hg) and reflects the arterial pressure resulting from the ejection of blood during ventricular contraction, or systole. The “diastolic arterial blood pressure” (DAP) is the lower value (usually about 80 mm Hg) and represents the arterial pressure of blood during ventricular relaxation, or diastole.

As used herein, “mean arterial pressure” (MAP) refers to an average blood pressure in an individual during a single cardiac cycle, that is, the average force driving blood into vessels that serve the tissues (see, e.g., Zheng L, et al. (July 2008), Stroke. 39 (7): 1932-7). Mean is a statistical concept and is calculated by taking the sum of the values divided by the number of values. Although complicated to measure directly and complicated to calculate, MAP can be approximated by adding the diastolic pressure to one-third of the pulse pressure or systolic pressure minus the diastolic pressure. Normally, the MAP falls within the range of 70-110 mm Hg. If the value falls below 60 mm Hg for an extended time, blood pressure will not be high enough to ensure circulation to and through the tissues, which results in ischemia, or insufficient blood flow. A condition called hypoxia, inadequate oxygenation of tissues, commonly accompanies ischemia. The term hypoxemia refers to low levels of oxygen in systemic arterial blood. Neurons are especially sensitive to hypoxia and may die or be damaged if blood flow and oxygen supplies are not quickly restored.

As used herein, “pulse pressure” (PP) refers to the difference between the measured systolic and diastolic pressures. The up and down fluctuation of the arterial pressure results from the pulsatile nature of the cardiac output, i.e., the heartbeat. Pulse pressure is determined by the interaction of the stroke volume of the heart, the compliance (ability to expand) of the arterial system and the resistance to flow in the arterial tree. By expanding under pressure, the aorta absorbs some of the force of the blood surge from the heart during a heartbeat. In this way, the pulse pressure is reduced from what it would be if the aorta were not compliant. The loss of arterial compliance that occurs with aging explains the elevated pulse pressures found in elderly patients.

“Ventricular pressure” is a measure of blood pressure within the ventricles of the heart. The pressure generated in the ventricular chambers of the heart can be measured and used in a variety of ways to understand changes in cardiac function. “Left ventricular pressure” (LVP) in conjunction with ventricular volume measurements has a history of being used to characterize the pressure-volume relationship in the beating heart. In addition, LVP is studied to ensure drugs developed do not negatively alter cardiac contractility. Left ventricular dP/dt is the first derivative of LVP, which is computed by software algorithms using calculus. Its peak value, dP/dtₘₐₓ, is a common, robust and sensitive indicator of changes in cardiac contractility if experimental parameters such as preload, afterload and heart rate are well controlled.

As used herein, cardiac “contractility” is an intrinsic property of the heart muscle that affects the heart's performance and can be modified by the autonomic system, circulating hormones, drugs and disease. Evaluation of a drug's effects on contractility is important in safety assessment studies, since either an increase or a decrease may be harmful under certain clinical situations. An increase in contractility dramatically increases the heart's energy consumption, which translates to increased oxygen consumption and increased coronary blood flow. This can have serious consequences in the presence of heart disease and/or coronary insufficiency. A decrease in contractility in an already diseased heart can exacerbate the symptoms and consequences of clinical heart failure.

The methods of treatment described herein employ administration to a subject (such as a human) an NPP1 protein, active fragments or fusion protein as described herein, in order to cure, delay, reduce the severity of, or ameliorate one or more symptoms of a cardiovascular disorder, or to prolong the survival of a subject beyond that expected in the absence of such treatment.

The term “treating” includes the application or administration of the NPP1 proteins, active fragments and fusion proteins of the invention to a subject, or application or administration of NPP1 proteins, active fragments and fusion proteins of the invention to a subject who has a cardiovascular disorder, such as hypertension, with the purpose of curing, healing, alleviating, relieving, altering, remedying, ameliorating, preventing, improving, or affecting the cardiovascular disease or disorder. The term “treating” refers to any indicia of success in the treatment or amelioration of an injury, pathology or condition, including any objective or subjective parameter such as abatement; remission; diminishing of symptoms or making the injury, pathology or condition more tolerable to the subject; slowing in the rate of degeneration or decline; making the final point of degeneration less debilitating; or improving a subject's physical or mental well-being. Treatment may be therapeutic or prophylactic. The treatment or amelioration of symptoms can be based on objective or subjective parameters; including the results of a physical examination.

### 1. Methods of Treatment

The present invention relates to uses of an isolated recombinant human soluble NPP1 (“sNPP1”) which lacks an N-terminal portion (i.e., lacking cytosolic and transmembrane domains) and fusion proteins thereof for the treatment of a cardiovascular disorder, such as hypertension.

In one embodiments, methods for improving cardiovascular function in a human patient are provided, the method comprising administering to the patient one or more doses of a recombinant human soluble ectonucleotide pyrophosphatase phosphodiesterase (hsNPP1), active fragment or fusion protein thereof.

In one embodiment, the improvement is a reduction in elevated blood pressure (e.g., reduction of blood pressure higher than 140 over 90 millimeters of mercury (mmHg) to within a normal blood pressure range (about 120 over 80 mm of mercury (mmHg)). In another embodiment, the improvement is a reduction in elevated blood pressure by at least 5 (e.g., at least 10, 15, 20, 25, 30, 35, 40, 45, 50, 55, 60, 65, or 70) %. In another embodiment, the improvement is a reduction in elevated blood pressure by about 1.5-fold, 2-fold, 2.5-fold, 3-fold, 3.5-fold, or 4-fold. In another embodiment, the improvement is a normalization of blood pressure (e.g., to about 120 over 80 mm of mercury (mmHg)). In another embodiment, the improvement is a reduction in left ventricular end-diastolic pressure (EDP) and end-systolic pressure (ESP) (e.g., by at least 5, 10, 15, 20, 25, 30, 35, 40, 45, 50, 55, 60, 65, or 70%). In another embodiment, the improvement is a reduction in left ventricular EDP and ESP by about 1.5-fold, 2-fold, 2.5-fold, 3-fold, 3.5-fold, or 4-fold. In another embodiment, the improvement is a reduction in ventricle stiffness (EDPVR) (e.g., by at least 5, 10, 15, 20, 25, 30, 35, 40, 45, 50, 55, 60, 65, or 70%). In another embodiment, the improvement is a reduction in ventricle stiffness by about 1.5-fold, 2-fold, 2.5-fold, 3-fold, 3.5-fold, or 4-fold. In another embodiment, the improvement is in increase in contractility (PRSW) (e.g., by at least 5, 10, 15, 20, 25, 30, 35, 40, 45, 50, 55, 60, 65, or 70%). In another embodiment, the improvement is in increase in contractility by about 1.5-fold, 2-fold, 2.5-fold, 3-fold, 3.5-fold, or 4-fold.

In another embodiment, methods for treating a human patient having a cardiovascular disorder are provided, the method comprising administering to the patient one or more doses of a recombinant human soluble ectonucleotide pyrophosphatase phosphodiesterase (hsNPP1), active fragment or fusion protein thereof. Exemplary cardiovascular diseases include, but are not limited to, coronary artery diseases (CAD) (such as angina and myocardial infarction (commonly known as a heart attack), stroke, heart failure, hypertensive heart disease, rheumatic heart disease, cardiomyopathy, heart arrhythmia, congenital heart disease, valvular heart disease, carditis, aortic aneurysms, peripheral artery disease, thromboembolic disease, and venous thrombosis.

In another embodiment, methods for treating a human patient having hypertension are provided, the method comprising administering to the patient one or more doses of a recombinant human soluble ectonucleotide pyrophosphatase phosphodiesterase (hsNPP1), active fragment or fusion protein thereof.

In another embodiment, methods of treating a human patient having hypertension are provided, the method comprising: a) identifying a human patient as having hypertension and b) administering to the identified patient one or more doses of a recombinant human soluble ectonucleotide pyrophosphatase phosphodiesterase (hsNPP1), active fragment or fusion protein thereof.

In another embodiment, methods for reducing hypertension in a human patient are provided, the method comprising administering to the patient one or more doses of a recombinant human soluble ectonucleotide pyrophosphatase phosphodiesterase (hsNPP1), active fragment or fusion protein thereof.

In another embodiment, the methods described herein result in a reduction in the patient's blood pressure (e.g., from an elevated blood pressure higher than about 140 over 90 millimeters of mercury (mmHg) to within a normal blood pressure range (about 120 over 80 mm of mercury (mmHg)). In another embodiment, the patient's blood pressure is reduced by at least 5 (e.g., at least 10, 15, 20, 25, 30, 35, 40, 45, 50, 55, 60, 65, or 70) % after treatment. In another embodiment, the patient's blood pressure is reduced by about 1.5-fold, 2-fold, 2.5-fold, 3-fold, 3.5-fold, or 4-fold after treatment. In another embodiment, the patient's blood pressure is normalized after treatment (e.g., to about 120 over 80 mm of mercury (mmHg)).

Generally, the dosage of fusion protein administered to a subject will vary depending upon known factors such as age, health and weight of the recipient, type of concurrent treatment, frequency of treatment, and the like. Usually, a dosage of active ingredient (i.e., fusion protein) can be between about 0.0001 and about 50 milligrams per kilogram of body weight. Precise dosage, frequency of administration and time span of treatment can be determined by a physician skilled in the art of administration of therapeutic proteins.

As defined herein, a therapeutically effective amount of protein (i.e., an effective dosage) ranges from about 0.001 to 50 mg/kg body weight. The skilled artisan will appreciate that certain factors may influence the dosage required to effectively treat a subject, including but not limited to the severity of the disease, previous treatments, the general health and/or age of the subject, and other diseases present. Moreover, treatment of a subject with a therapeutically effective amount of protein can include a single treatment or, preferably, can include a series of treatments. It will also be appreciated that the effective dosage of protein used for treatment may increase or decrease over the course of a particular treatment.

As defined herein, a therapeutically effective amount of protein or polypeptide (i.e., an effective dosage) ranges from about 0.001 to 50 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight. In one embodiment, the hsNPP1 is administered in one or more doses containing about 0.1 mg/kg, 0.2 mg/kg, 0.3 mg/kg, 0.4 mg/kg, 0.5 mg/kg, 1.0 mg/kg, 2.0 mg/kg, 3.0 mg/kg, 4.0 mg/kg, 5.0 mg/kg, 6.0 mg/kg, 7.0 mg/kg, 8.0 mg/kg, 9.0 mg/kg, 10.0 mg/kg, 11.0 mg/kg, 12.0 mg/kg, 13.0 mg/kg, 14.0 mg/kg, 15.0 mg/kg, 16.0 mg/kg, 17.0 mg/kg, 18.0 mg/kg, 19.0 mg/kg, 20.0 mg/kg, 21.0 mg/kg, 22.0 mg/kg, 23.0 mg/kg, 24.0 mg/kg, 25.0 mg/kg, 26.0 mg/kg, 27.0 mg/kg, 28.0 mg/kg, 29.0 mg/kg, 30.0 mg/kg, 31.0 mg/kg, 32.0 mg/kg, 33.0 mg/kg, 34.0 mg/kg, 35.0 mg/kg, 36.0 mg/kg, 37.0 mg/kg, 38.0 mg/kg, 39.0 mg/kg, 40.0 mg/kg, 41.0 mg/kg, 42.0 mg/kg, 43.0 mg/kg, 44.0 mg/kg, or 45.0 mg/kg. In another embodiment, about 0.5 to about 30 mg, about 0.5 to about 20 mg, about 0.5 to about 10 mg, or about 0.5 to about 5 mg are administered to the patient. In another embodiment, the hsNPP1 is administered in one or more doses containing about 1.0 mg/kg to about 5.0 mg/kg hsNPP1. In another embodiment, the hsNPP1 is administered in one or more doses containing about 1.0 mg/kg to about 10.0 mg/kg hsNPP1. The skilled artisan will appreciate that certain factors may influence the dosage required to effectively treat a subject, including, but not limited to, the severity of the disease or disorder, previous treatments, the general health and/or age of the subject, and other diseases present.

Moreover, treatment of a subject with a therapeutically effective amount of a protein, polypeptide, or antibody can include a single treatment or, preferably, can include a series of treatments. In one embodiment, in the range of between about 0.1 to 20 mg/kg body weight, one time per week, twice per week, once in about 10 days, once in about 12 days, once in about 14 days, once in about 17 days, once in about 20 days, once in about 25 days, or once in about 30 days. In one embodiment, the time period between doses of the hsNPP1 is at least 2 days and can be longer, for example at least 3 days, at least 1 week, 2 weeks or 1 month. In another embodiment, the therapeutically effective dose of sNPP1, biologically active fragment or fusion protein thereof is administered to a patient between one time every 5 days and one time every 30 days for a period of time determined by a practitioner of skill in the art of medical sciences. In another embodiment, the period of time will be the remainder of the patient's life span. In another embodiment, the dosing frequency is between one time every 5 days and one time every 25 days. In another embodiment, the dosing frequency is between one time every 5 days and one time every 21 days. In another embodiment, the dosing frequency is between one time every 7 days and one time every 14 days. hsNPP1, biologically active fragment or fusion protein thereof can be administered one time every 5 days, one time every 6 days, one time every 7 days, one time every 8 days, one time every 9 days, one time every 10 days, one time every 11 days, one time every 12 days, one time every 13 days, or one time every 14 days. In some embodiments, hsNPP1, biologically active fragment or fusion protein thereof is administered about weekly. In other embodiments, sNPP1, biologically active fragment or fusion protein thereof is administered about bi-weekly. In one embodiment, the dosing frequency is one time about 30 days. It will also be appreciated that the effective dosage of soluble sNPP1 protein, biologically active fragment or fusion protein thereof used for the treatment may increase or decrease over the course of a particular treatment.

In one embodiment, about 1 mg/kg of hsNPP1, biologically active fragment or fusion protein is administered to the patient once a week. In one embodiment, about 2 mg/kg of hsNPP1, biologically active fragment or fusion protein is administered to the patient once a week. In one embodiment, about 3 mg/kg of hsNPP1, biologically active fragment or fusion protein is administered to the patient once a week. In one embodiment, about 4 mg/kg of sNPP1, biologically active fragment or fusion protein is administered to the patient once a week. In one embodiment, about 5 mg/kg of hsNPP1, biologically active fragment or fusion protein is administered to the patient once a week. In one embodiment, about 6 mg/kg of hsNPP1, biologically active fragment or fusion protein is administered to the patient once a week. In one embodiment, about 7 mg/kg of hsNPP1, biologically active fragment or fusion protein is administered to the patient once a week. In one embodiment, about 8 mg/kg of hsNPP1, biologically active fragment or fusion protein is administered to the patient once a week. In one embodiment, about 9 mg/kg of hsNPP1, biologically active fragment or fusion protein is administered to the patient once a week. In one embodiment, about 10 mg/kg of hsNPP1, biologically active fragment or fusion protein is administered to the patient once a week.

hsNPP1, biologically active fragment or fusion protein can be administered by, for example, subcutaneous injections, intramuscular injections, and intravenous (IV) infusions or injections.

In one embodiment, hsNPP1, biologically active fragment or fusion protein is administered intravenously by IV infusion by any useful method. In one example, hsNPP1, biologically active fragment or fusion protein can be administered by intravenous infusion through a peripheral line. In another example, hsNPP1, biologically active fragment or fusion protein can be administered by intravenous infusion through a peripherally inserted central catheter.

In another embodiment, hsNPP1, biologically active fragment or fusion protein is administered intravenously by IV injection. In another embodiment, hsNPP1, biologically active fragment or fusion protein is administered via intraperitoneal injection. In another embodiment, hsNPP1, biologically active fragment or fusion protein is administered by subcutaneous injections. In another embodiment, hsNPP1, biologically active fragment or fusion protein is administered by intramuscular injections.

In still another embodiment, hsNPP1, biologically active fragment or fusion protein is administered via a pharmaceutically acceptable capsule of the therapeutic protein. For example, the capsule can be an enteric-coated gelatin capsule.

In one embodiment, the method involves administering the soluble NPP1 protein or NPP1 fusion protein of the invention alone, or in combination with other agent(s). Exemplary therapeutic agents include, but are not limited to a thiazide diuretic (e.g., hydrochlorothiazide (Microzide) or chlorthalidone), a beta blocker (e.g., acebutolol (Sectral) or atenolol (Tenormin)), an angiotensin-converting enzyme (ACE) inhibitor (e.g., lisinopril (Zestril), benazepril (Lotensin), or captopril (Capoten)), an angiotensin II receptor blocker (ARB) (e.g., candesartan (Atacand) or losartan (Cozaar)), a calcium channel blocker (e.g., amlodipine (Norvasc) or diltiazem (Cardizem)), a renin inhibitor (e.g., Aliskiren (Tekturna)), an alpha blocker (e.g., doxazosin (Cardura) or prazosin (Minipress)), an alpha-beta blocker (e.g., carvedilol (Coreg) or labetalol (Trandate)), a central-acting agent (e.g., clonidine (Catapres, Kapvay), guanfacine (Intuniv, Tenex) and methyldopa), a vasodilator (e.g., hydralazine and minoxidil), and/or an aldosterone antagonist (e.g., spironolactone (Aldactone) or eplerenone (Inspra)).

In one embodiment, the isolated sNPP1 proteins, fragments, and fusion proteins can be administered before, after or concurrently with the agent or can be co-administered with other known therapies. Co-administration of the isolated sNPP1 proteins, fragments, and fusion proteins of the present invention with other therapeutic agents may provide two agents which operate via different mechanisms which yield an increased therapeutic effect. Such co-administration can solve problems due to development of resistance to drugs.

### 2. sNPP1

The present invention employs soluble NPP1 (e.g., hsNPP1) that has a biologically active NPP1 domain of NPP1 (i.e., NPP1 components that contain at least one extracellular catalytic domain of naturally occurring NPP1 for the pyrophosphatase and/or phosphodiesterase activity). The soluble NPP1 proteins of the invention comprise at least the NPP1 domain essential to carry out the pyrophosphatase and/or phosphodiesterase activity.

In one embodiment, the soluble NPP1, fragment, and fusion proteins thereof can form functional homodimers or monomer. In another embodiment, a soluble NPP1 protein or NPP1 fusion protein thereof can be assayed for pyrophosphatase activity as well as the ability to increase pyrophosphate levels in vivo.

Described herein are various amino acid sequences of soluble NPP1 compounds, fusion partners and fusion proteins that are suitable for use according to the methods provided herein. SEQ ID NO:5 shows the amino acid sequences of a soluble NPP1 containing amino acids from 107 to 925 of SEQ ID NO:1. SEQ ID NO:6 shows the amino acid sequence of a soluble NPP1 containing amino acids from 187 to 925 of SEQ ID NO:1. SEQ ID NO:7 shows the amino acid sequence of the Fc region of human IgG1 including the hinge region. SEQ ID NO:8 shows the amino acid sequence of the Fc of human IgG1 including a partial hinge region. SEQ ID NO:9 shows the amino acid sequence of a NPP1-Fc fusion protein. The NPP1 component contains SEQ ID NO:5, and the Fc sequence includes the hinge region. SEQ ID NO:10 shows the amino acid sequence of a NPP1-Fc fusion protein. The soluble NPP1 contains SEQ ID NO:5, and the Fc sequence includes the partial hinge region. SEQ ID NO:1 shows the amino acid sequence of a NPP1-Fc fusion protein. The soluble NPP1 contains SEQ ID NO:6, and the Fc sequence includes the hinge region. SEQ ID NO:12 shows the amino acid sequence of a NPP1-Fc fusion protein. The soluble NPP1 contains SEQ ID NO:6, and the Fc sequence includes the partial hinge region.

Preferred soluble NPP1 proteins and NPP1 fusion proteins of the invention are enzymatically active in vivo (e.g., human). In one embodiment, the soluble protein comprises amino acid sequence having at least 60, 70, 75, 80, 85, 90, 91, 92, 93, 94, 95, 96, 97, 98, 99% sequence identity to the following sequence:

(SEQ ID NO: 2)
PSCAKEVKSCKGRCFERTFGNCRCDAACVELGNCCLDYQETCIEPEHIWT
CNKFRCGEKRLTRSLCACSDDCKDKGDCCINYSSVCQGEKSWVEEPCESI
NEPQCPAGFETPPTLLFSLDGFRAEYLHTWGGLLPVISKLKKCGTYTKNM
RPVYPTKTFPNHYSIVTGLYPESHGIIDNKMYDPKMNASFSLKSKEKFNP
EWYKGEPIWVTAKYQGLKSGTFFWPGSDVEINGIFPDIYKMYNGSVPFEE
RILAVLQWLQLPKDERPHFYTLYLEEPDSSGHSYGPVSSEVIKALQRVDG
MVGMLMDGLKELNLHRCLNLILISDHGMEQGSCKKYIYLNKYLGDVKNIK
VIYGPAARLRPSDVPDKYYSFNYEGIARNLSCREPNQHFKPYLKHFLPKR
LHFAKSDRIEPLTFYLDPQWQLALNPSERKYCGSGFHGSDNVFSNMQALF
VGYGPGFKHGIEADTFENIEVYNLMCDLLNLTPAPNNGTHGSLNHLLKNP
VYTPKHPKEVHPLVQCPFTRNPRDNLGCSCNPSILPIEDFQTQFNLTVAE
EKIIKHETLPYGRPRVLQKENTICLLSQHQFMSGYSQDILMPLWTSYTVD
RNDSFSTEDFSNCLYQDFRIPLSPVHKCSFYKNNTKVSYGFLSPPQLNKN
SSGIYSEALLTTNIVPMYQSFQVIWRYFHDTLLRKYAEERNGVNVVSGPV
FDFDYDGRCDSLENLRQKRRVIRNQEILIPTHFFIVLTSCKDTSQTPLHC
ENLDTLAFILPHRTDNSESCVHGKHDSSWVEELLMLHRARITDVEHITGL
SFYQQRKEPVSDILKLKTHLPTFSQED

SEQ ID NO:2 is the amino acid sequence of a sNPP1 that contains the cysteine-rich region, catalytic region and c-terminal region.

Any desired enzymatically active form of soluble NPP1 can be used in the methods described herein. The enzymatically active sNPP1 can increase pyrophosphate (PPi) levels in suitable enzymatic assays, and can be assayed for pyrophosphatase activity, phosphodiesterase activity, or pyrophosphatase and phosphodiesterase activity. Typically, the sNPP1 contains at least an NPP1 component that lacks the N-terminal cytosolic and transmembrane domains of naturally occurring transmembrane NPP1.

SEQ ID NO:1 is the amino acid sequence of wild-type NPP1 protein. The cytosolic and transmembrane regions are underlined. The potential N-glycosylation sites are in bold. The amino acid motif “PSCAKE” (SEQ ID NO:17) in bold is the start of a soluble NPP1 which includes the cysteine rich region.

In preferred aspects, the NPP1 component contains the cysteine-rich region (amino acids 99-204 of SEQ ID NO:1) and the catalytic region (amino acids 205-591 of SEQ ID NO:1) of naturally occurring human NPP1. Typically, the NPP1 component also includes the C-terminal region (amino acids 592 to 925 of SEQ ID NO:1), and has the amino acid sequence of SEQ ID NO:2. However, the C-terminal region can be truncated if desired. Accordingly, preferred NPP1 components include the cysteine-rich region and catalytic region of human NPP1 (amino acids 99-591 of SEQ ID NO:1) or the cysteine-rich region, the catalytic region and the C-terminal region of human NPP1 (SEQ ID NO:2). Other preferred NPP1 components contain only a portion of the cysteine-rich domain and have the sequence of amino acids 107 to 925 of SEQ ID NO:1 or amino acids 187 to 925 of SEQ ID NO:1. The cysteine rich region of NPP1 (i.e., amino acids 99 to 204 of SEQ ID NO: 1) can facilitate dimerization of the sNPP1. The sNPP1, including fusion proteins, can be in the form of a monomer of functional homodimer.

The amino acid sequence of the NPP1 component can be a variant of the naturally occurring NPP1 sequence, provided that the NPP1 component is enzymatically active. NPP1 variants are enzymatically active and have at least 80%, at least 85%, at least 90%, at least 95% and more preferably at least 96% amino acid sequence identity to the corresponding portion of human NPP1 (e.g., over the length of the cysteine-rich region, the catalytic region, the c-terminal region, the cysteine-rich region plus the catalytic region, the cystein-rich region plus the catalytic region plus the c-terminal region. Preferred NPP1 variants have at least 90%, preferably at least 95%, more preferably at least 97% amino acid sequence identity to (i) the amino acid sequence of residues 205-591 of SEQ ID NO: 1, (ii) the amino acid sequence of residues 99-591 of SEQ ID NO:1, (iii) the amino acid sequence of residues 99-925 of SEQ ID NO:1, (iv) the amino acid sequence of residues 107-925 of SEQ ID NO:1, or (v) the amino acid sequence of residues 187-925 of SEQ ID NO:1. Suitable positions for amino acid variation are well-known from NPP1 structural studies and analysis of disease-associated mutations in NPP1. For example, substitution of the following amino acids occurs in certain disease-associated mutations that reduce NPP1 enzymatic activity, and variations of the amino acids at these positions should be avoided: Ser216, Gly242, Pro250, Gly266, Pro305, Arg349, Tyr371, Arg456, Tyr471, His500, Ser504, Tyr513, Asp538, Tyr570, Lys579, Gly586; Tyr659, Glu668, Cys726, Arg774, His777, Asn792, Asp804, Arg821, Arg888, and Tyr901. (See, e.g., Jansen, S. et al., Structure 20:1948-1959 (2012)).

In one embodiment, the soluble NPP1 protein can be a fusion protein recombinantly fused or chemically bonded (e.g., covalent bond, ionic bond, hydrophobic bond and Van der Waals force) to a fusion partner. In another embodiment, the fusion protein has at least 70, 75, 80, 85, 90, 91, 92, 93, 94, 95, 96, 97, 98, or 99% sequence identity to SEQ ID NO: 3 or SEQ ID NO:4. SEQ ID NO:4 is the amino acid sequence of sNPP1-Fc-D10 (SEQ ID NO:4). The Fc sequence is underlined.

To determine the percent identity of two amino acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, or 90% of the length of the reference sequence (e.g., sNPP1 amino acid sequence of SEQ ID NO:2; amino acids 107-925 of SEQ ID NO:1 or amino acids 187-925 of SEQ ID NO:1). The amino acid residues or nucleotides at corresponding amino acid positions are then compared. When a position in the first sequence is occupied by the same amino acid residue or nucleotide as the corresponding position in the second sequence, then the molecules are identical at that position (as used herein amino acid is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch (J Mol Biol 1970, 48, 444-453) algorithm which has been incorporated into the GAP program in the GCG software package (available at www.gcg.com), using either a Blosum 62 matrix or a PAM250 matrix, and a gap weight of 16, 14, 12, 10, 8, 6, or 4 and a length weight of 1, 2, 3, 4, 5, or 6. In another embodiment, the percent identity between two amino acid is determined using the algorithm of E. Meyers and W. Miller (CABIOS, 1989, 4, 11-17) which has been incorporated into the ALIGN program (version 2.0 or 2.0U), using a PAM120 weight residue table, a gap length penalty of 12 and a gap penalty of 4.

The sNPP1 can consist of or consist essentially of an NPP1 component as described herein. Alternatively, the sNPP1 can be in the form of a fusion protein that contains an NPP1 component and one or more other polypeptides, referred to as fusion partners, optionally through a suitable linker in each instance, or in the form of a conjugate between an NPP1 component and another molecule (e.g., PEG). When the sNPP1 is in the form of a fusion protein, each fusion partner is preferably located c-terminally to the NPP1 component. Without wishing to be bound by any particular theory, it is believed that fusion proteins that contain an NPP1 component that contains the cysteine-rich region and catalytic region, and one or more fusion proteins that are located c-terminally to the NPP1 component, are preferred over other configurations of NPP1 fusion proteins because they can be expressed at sufficient levels and are sufficiently stable to be used as therapeutic proteins.

Any suitable fusion partner can be included in the fusion protein. Advantageously, a number of fusion partners are well-known in the art that can provide certain advantages, such as reduced aggregation and immunogenicity, increased the solubility, improved expression and/or stability, and improved pharmacokinetic and/or pharmacodynamics performance. See, e.g., Strohl, W. R. BioDrugs 29:215-239 (2015). For example, it is well-known that albumin, albumin fragments or albumin variants (e.g., human serum albumin and fragments or variants thereof) can be incorporated into fusion proteins and that such fusion proteins can be easily purified, stable and have an improved plasma half-life. Suitable albumin, albumin fragments and albumin variants that can be used in the sNPP1 fusion proteins are disclosed, for example in WO 2005/077042A2 and WO 03/076567A2, each of which is incorporated herein by reference in its entirety. Fusions to human transferrin are also known to improve half-life. See, e.g., Kim B J et al., J Pharmacol Expr Ther 334 (3): 682-692 (2010); and WO 2000/020746. Peptides that bind to albumin or transferrin, such as antibodies or antibody fragments, can also be used. See, e.g., EP 0486525 B1, U.S. Pat. No. 6,267,964 B1, WO 04/001064A2, WO 02/076489A1, WO 01/45746, WO 2006/004603, and WO 2008/028977. Similarly immunoglobulin Fc fusion proteins are well-known. See, e.g., Czajkowsky D M et al., EMBO Mol Med 4 (10): 1015-1028 (2012), U.S. Pat. Nos. 7,902,151; and 7,858,297, the entire teachings of which are incorporated herein by reference in their entirety. The fusion protein can also include a CTP sequence (see also, Fares et al., Endocrinol 2010, 151, 4410-4417; Fares et al., Proc Natl Acad Sci 1992, 89, 4304-4308; and Furuhashi et al., Mol Endocrinol 1995, 9, 54-63). Preferably, the fusion partner is the Fc of an immunoglobulin (e.g., Fc or human IgG1). The Fc can include CH1, CH2 and CH3 of human IgG1, and optionally the human IgG1 hinge region (EPKSCDKTHTCPPCP (SEQ ID NO:13)) or a portion of the human IgG1 hinge region (e.g., DKTHTCPPCP (SEQ ID NO:14) or PKSCDKTHTCPPCP (SEQ ID NO:15)) if desired. In some fusion proteins, the Fc can include CH2 and CH3 of human IgG1, or the Fc of human IgG2 or human IgG4, if desired. Preferably, the sNPP1 fusion protein comprises an NPP1 component and a peptide that increases the half-life of the fusion protein, most preferably the Fc of an immunoglobulin (e.g., Fc or human IgG1). As used herein, a “protein that increases the half-life of the fusion protein” refers to a protein that, when fused to a soluble NPP1 or biologically active fragment, increases the half-life of the soluble NPP1 polypeptide or biologically active fragment as compared to the half-life of the soluble NPP1 polypeptide, alone, or the NPP1 biologically active fragment, alone. In one embodiment, the half-life of the NPP1 fusion protein is increased 50% as compared to the half-life of the NPP1 polypeptide or biologically active fragment, alone. In another embodiment, the half-life of the NPP1 fusion protein is increased 60% as compared to the half-life of the NPP1 polypeptide or biologically active fragment, alone. In another embodiment, the half-life of the NPP1 fusion protein is increased 70% as compared to the half-life of the NPP1 polypeptide or biologically active fragment, alone. In another embodiment, the half-life of the NPP1 fusion protein is increased 80% as compared to the half-life of the NPP1 polypeptide or biologically active fragment, alone. In another embodiment, the half-life of the NPP1 fusion protein is increased 90% as compared to the half-life of the NPP1 polypeptide or biologically active fragment, alone.

In another embodiment, the half-life of the NPP1 fusion protein is increased 2 fold, 3 fold, 4 fold, 5 fold, 6 fold, 7 fold, 8 fold, 9 fold, or 10 fold as compared to the half-life of the NPP1 polypeptide or biologically active fragment, alone. Methods for determining the half-life of a protein or fusion protein are well known in the art. For example, Zhou et al., Determining Protein Half-Lives, Methods in Molecular Biology 2004, 284, 67-77 discloses numerous methods for testing of the half-life of a protein. If desired, the fusion protein can be conjugated to polymers or other suitable compounds that extend half-life, such as polyethylene glycol (PEG), can be conjugated to the NPP1 fusion proteins.

In one embodiment, the peptide which increases the half-life of the fusion protein is a CTP sequence (see also, Fares et al., 2010, Endocrinol., 151(9):4410-4417; Fares et al., 1992, Proc. Natl. Acad. Sci, 89 (10):4304-4308; and Furuhashi et al., 1995, Molec. Endocrinol., 9(1):54-63).

In another embodiment, the peptide which increases the half-life of the fusion protein is an Fc domain of an Ig.

Fusion partners may also be selected to target the fusion protein to desired sites of clinical or biological importance (e.g., site of calcification). For example, peptides that have high affinity to the bone are described in U.S. Pat. No. 7,323,542, the entire teachings of which are incorporated herein by reference. Peptides that can increase protein targeting to calcification sites can contain a consecutive stretch of at least about 4 acidic amino acids, for example, glutamic acids or aspartic acids. Typically, the peptide that targets the fusion protein to calcification sites will comprise between 4 and 20 consecutive acidic amino acids, for example 4, 5, 6, 7, 8, 9, 10, 11, 12, 13, 14, 15, 16, 17, 18, 19 or 20 consecutive amino acids selected from glutamic acid and aspartic acid. The peptide can consist solely of glutamic acid residues, solely of aspartic acid residues, or be a mixture of glutamic acid and aspartic acid residues. A particularly preferred moiety for targeting to sights of calcification is Aspio (SEQ ID NO:18).

In one embodiment, the NPP1 fusion protein of the invention comprises an NPP1 polypeptide and a moiety that increase protein targeting to calcification sites such as a consecutive stretch of acidic amino acids, for example, glutamic acids or aspartic acids.

Suitable peptide linkers for use in fusion proteins are well-known and typically adopt a flexible extended conformation and do not interfere with the function of the NPP1 component or the fusion partners. Peptide linker sequences may contain Gly, His, Asn and Ser residues in any combination. The useful peptide linkers include, without limitation, poly-Gly, poly-His, poly-Asn, or poly-Ser. Other near neutral amino acids, such as Thr and Ala can be also used in the linker sequence. Amino acid sequences which can be usefully employed as linkers include those disclosed in Maratea et al., Gene 1985, 40, 39-46; Murphy et al., Proc Natl Acad Sci USA 1986, 83, 8258-8262; U.S. Pat. Nos. 4,935,233 and 4,751,180. Other suitable linkers can be obtained from naturally occurring proteins, such as the hinge region of an immunoglobulin. A preferred synthetic linker is (Gly₄Ser)ₙ, where n is 1, 2, 3, 4, 5, 6, 7, 8, 9 or 10 (SEQ ID NO:19). Preferably, n is 3 or 4. For example, in some embodiments the linker is (Gly₄Ser)₃ (SEQ ID NO:16) and the fusion protein include a linker with the amino acid sequence GlyGlyGlyGlySerGlyGlyGlyGlySerGlyGlyGlyGlySer (SEQ ID NO:16). Typically, the linker is from 1 to about 50 amino acid residues in length, or 1 to about 25 amino acids in length. Frequently, the linker is between about 8 and about 20 amino acids in length. Preferred NPP1 fusion proteins comprise from N-terminus to C-terminus an NPP1 component, optionally a linker, an Fc region of an immunoglobulin (e.g., human IgG1 Fc optionally including hinge or a portion thereof), optionally a second liner, and optionally a targeting moiety. Thus, the Fc region and the optional targeting moiety, when present, are cach located C-terminally to the NPP1 component. The NPP1 component preferably comprises the cysteine-rich region and the catalytic domain of NPP1, lacks the N-terminal cytosolic and transmembrane domains, and optionally contains the C-terminal region.

A preferred fusion protein comprises, from N-terminus to C-terminus, an NPP1 component comprising the cysteine-rich domain, the catalytic domain and the C-terminal region of human NPP1; and the Fc region, including hinge, of a human immunoglobulin. Preferably, the Fc region is from human IgG1. In particular embodiments, the fusion protein has at least 80%, at least 85%, at least 90%, at least 91%, at least 92%, at least 93%, at least 94%, at least 95%, at least 96%, at least 97%, at least 98%, or at least 99% sequence identity to SEQ ID NO:3. SEQ ID NO:3 is the amino acid sequence of sNPP1-Fc fusion protein.

A preferred fusion protein of this type has the amino acid sequence of SEQ ID NO:3.

Another preferred fusion protein comprises, from N-terminus to C-terminus, an NPP1 component comprising the cysteine-rich domain, the catalytic domain and the C-terminal region of human NPP1; a linker (e.g., (Gly₄Ser)₃ (SEQ ID NO:16)); and the Fc region, including hinge, of a human immunoglobulin. Preferably, the Fc region is from human IgG1.

Another preferred fusion protein comprises, from N-terminus to C-terminus, an NPP1 component comprising the cysteine-rich domain, the catalytic domain and the c-terminal region of human NPP1; the Fc region, including hinge or a portion thereof, of a human immunoglobulin; and a moiety targeting the fusion protein to sites of calcification. Preferably, the Fc region is from human IgG1. Preferably, the moiety targeting the fusion protein to sites of calcification is Aspio (SEQ ID NO:18). More preferably, the Fc region is from human IgG1 and the moiety targeting the fusion protein to sites of calcification is Aspio (SEQ ID NO:18). In particular embodiments, the fusion protein has at least 80%, at least 85%, at least 90%, at least 91%, at least 92%, at least 93%, at least 94%, at least 95%, at least 96%, at least 97%, at least 98%, or at least 99% sequence identity to SEQ ID NO:4. A preferred fusion protein of this type has the amino acid sequence of SEQ ID NO:4.

Another preferred fusion protein comprises, from N-terminus to C-terminus, an NPP1 component comprising the cysteine-rich domain, the catalytic domain and the c-terminal region of human NPP1; a linker (e.g., (Gly₄Ser)₃(SEQ ID NO:16)); the Fc region, including hinge or a portion thereof, of a human immunoglobulin; and a moiety targeting the fusion protein to sites of calcification. Preferably, the Fc region is from human IgG1. Preferably, the moiety targeting the fusion protein to sites of calcification is Aspio (SEQ ID NO:18). More preferably, the Fc region is from human IgG1 and the moiety targeting the fusion protein to sites of calcification is Asp₁₀ (SEQ ID NO:18).

Another preferred fusion protein comprises, from N-terminus to C-terminus, an NPP1 component comprising a portion of the cysteine-rich domain, the catalytic domain and the c-terminal region of human NPP1; optionally a linker (e.g., (Gly₄Ser)₃(SEQ ID NO:16)); the Fc region, including hinge or a portion thereof, of a human immunoglobulin. Preferably, the Fc region is from human IgG1. In particular embodiments, the fusion protein has at least 80%, at least 85%, at least 90%, at least 91%, at least 92%, at least 93%, at least 94%, at least 95%, at least 96%, at least 97%, at least 98%, or at least 99% sequence identity to SEQ ID NO:9, SEQ ID NO:10, SEQ ID NO:11, or SEQ ID NO:12. Preferred fusion protein of this type have the amino acid sequence of SEQ ID NO:9, SEQ ID NO:10, SEQ ID NO:11 or SEQ ID NO:12. In particularly preferred aspects, a fusion protein of SEQ ID NO:3 is administered in accordance with the methods described herein. In other particularly preferred aspect, a fusion protein of SEQ ID NO:4 is administered in accordance with in the methods described herein. In other particularly preferred aspect, a fusion protein of SEQ ID NO:9 is administered in accordance with in the methods described herein. In other particularly preferred aspect, a fusion protein of SEQ ID NO:10 is administered in accordance with the methods described herein. In other particularly preferred aspect, a fusion protein of SEQ ID NO:11 is administered in accordance with the methods described herein. In other particularly preferred aspect, a fusion protein of SEQ ID NO:12 is administered in accordance with the methods described herein.

Fusion proteins of the present invention can be prepared using standard methods, including recombinant techniques or chemical conjugation well known in the art. Techniques useful for isolating and characterizing the nucleic acids and proteins of the present invention are well known to those of skill in the art and standard molecular biology and biochemical manuals can be consulted to select suitable protocols for use without undue experimentation. See, for example, Sambrook et al., 1989, “Molecular Cloning: A Laboratory Manual”, 2^{nd} ed., Cold Spring Harbor, the content of which is herein incorporated by reference in its entirety. The isolated recombinant human sNPP1, fragment, and fusion proteins thereof, can be produced in any useful protein expression system including, without limitation, cell culture (e.g., CHO cells, COS cells, HEK203), bacteria such as Escherichia coli (E. coli) and transgenic animals, including, but no limited to, mammals and avians (e.g., chickens, quail, duck and turkey). For expression, a construct that encodes the sNPP1 and includes a suitable signal sequence (e.g, from human Ig heavy chain, NPP2, NPP4, NPP7 or human serum albumin, for example) in frame with the sequence of the sNPP1 and operably linked to suitable expression control elements.

The sNPP1, including the fusion proteins, and physiologically acceptable salt forms thereof are typically formulated into a pharmaceutical composition for administration in accordance with the methods described herein. Pharmaceutical compositions typically include a pharmaceutically acceptable carrier or excipient. Compositions comprising such carriers, including composite molecules, are formulated by well-known conventional methods (see, for example, Remington's Pharmaceutical Sciences, 14^{th} ed., Mack Publishing Co., Easton, PA), the entire teachings of which are incorporated herein by reference. The carrier may comprise a diluent. In one embodiment, the pharmaceutical carrier can be a liquid and the fusion protein may be in the form of a solution. The pharmaceutical carrier can be wax, fat, or alcohol. In another embodiment, the pharmaceutically acceptable carrier may be a solid in the form of a powder, a lyophilized powder, or a tablet. In one embodiment, the carrier may comprise a liposome or a microcapsule. The pharmaceutical compositions can be in the form of a sterile lyophilized powder for injection upon reconstitution with a diluent. The diluent can be water for injection, bacteriostatic water for injection, or sterile saline. The lyophilized powder may be produced by freeze drying a solution of the fusion protein to produce the protein in dry form. As is known in the art, the lyophilized protein generally has increased stability and a longer shelf life than a liquid solution of the protein.

### EQUIVALENTS

Those skilled in the art will recognize, or be able to ascertain using no more than routine experimentation, many equivalents of the specific embodiments described herein. Such equivalents are intended to be encompassed by the following claims. Any combination of the embodiments disclosed in the any plurality of the dependent claims or Examples is contemplated to be within the scope of the disclosure.

### INCORPORATION BY REFERENCE

The disclosure of each and every U.S. and foreign patent and pending patent application and publication referred to herein is specifically incorporated herein by reference in its entirety, as are the contents of Sequence Listing and Figures.

### EXAMPLES

The present invention is further exemplified by the following examples. The examples are for illustrative purpose only and are not intended, nor should they be construed as limiting the invention in any manner.

### Example 1: Evaluation of hEnpp1 Treatment on Cardiovascular Hemodynamics and Function in Asj-2J Mice

Experiments were conducted to determine if Enpp1 treatment improves elevated blood pressure and left ventricular hypertrophy in Asj-2J mice. Two week old Enpp1^{asj-2J} mice received 2100 U/kg of hEnpp1-Fc (TSAC 2.7) every other day (EOT) subcutaneously over the course of six weeks. Vehicle-treated wild-type mice and Asj-2j mice served as controls. At seven weeks of age, the mice were shipped to third party analyst for assessment of primary and secondary outcome measures. Dosing was continued by the analyst throughout the testing period (i.e., through week 11). Primary outcome measures included: echocardiogram (cardiac dimensions, heart rate (“HR”) and fractional shortening (“FS”), terminal hemodynamics (systolic arterial pressure (“SAP”), diastolic arterial pressure (“DAP”), mean arterial pressure (“MAP”), pulse pressure (“PP”), heart rate (“HR”), left ventricular pressure (“LVP”)), pressure volume loops (heart compliance and contractility), and aorta and vibrissae calcification. Secondary outcome measures included Enpp1 protein, pyrophosphate (PPi), and ADAs.

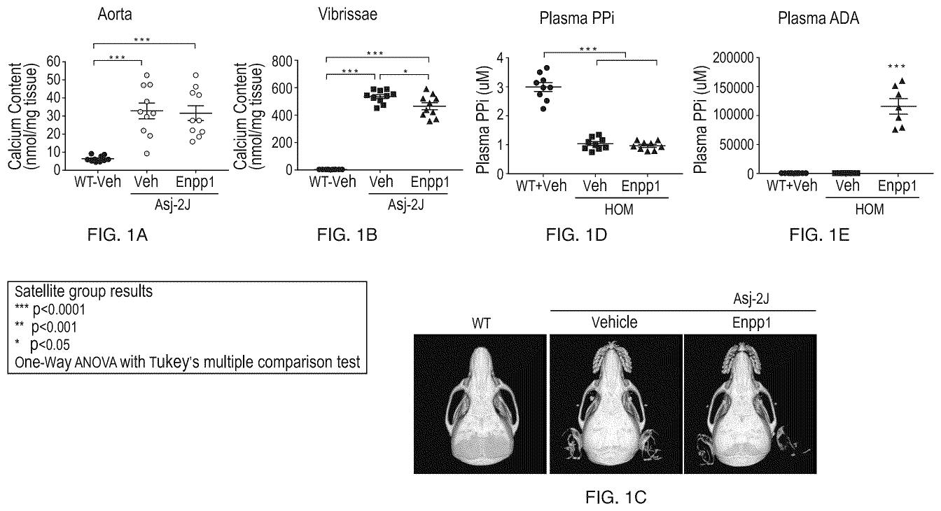

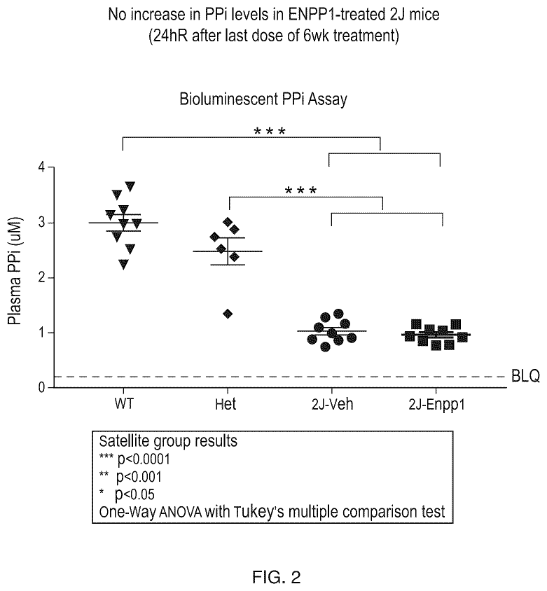

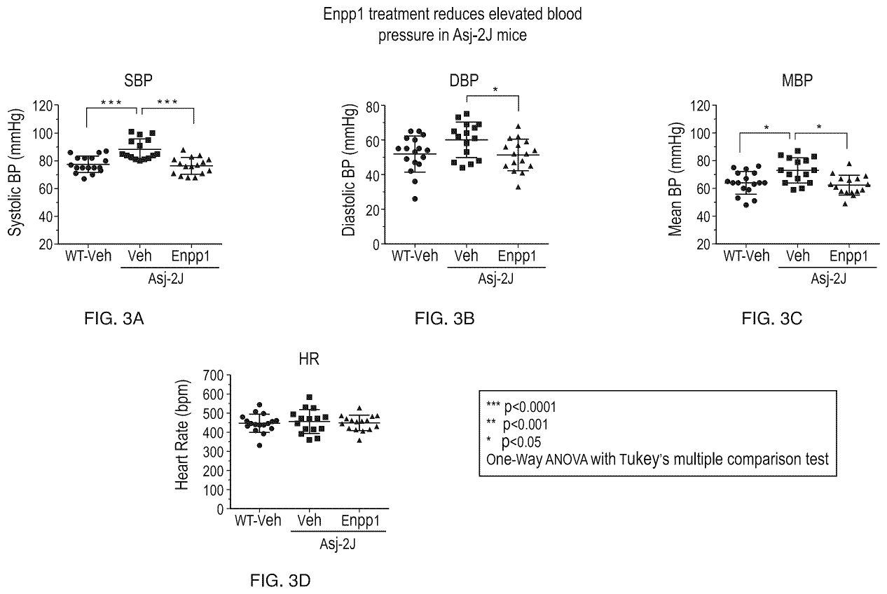

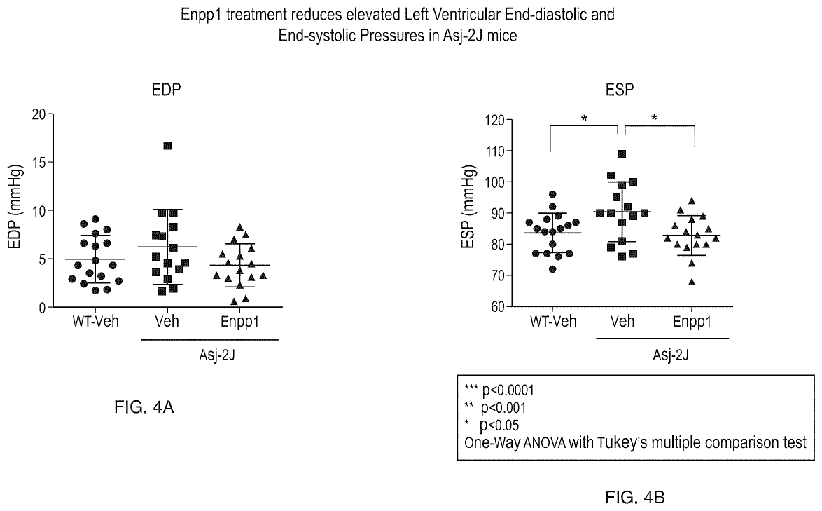

As shown in FIGS. 1A-1C, there was no reduction in calcification, no increase in plasma pyrophosphate (PPi) levels (FIG. 1D), and high plasma ADA levels (FIG. 1E) in ENPP 1-treated Asj-2J mice after six weeks of treatment. As shown in FIG. 2, there was no increase in PP1 levels in ENPP1-treated Asj-2J mice, twenty-four hours after the last dose of the six week treatment. However, as shown in FIGS. 3A-3D, ENPP1 treatment reduced elevated blood pressure in Asj-2J mice. In addition, as shown in FIGS. 4A-4B, ENPP1 treatment reduced elevated left ventricular end-diastolic and end-systolic pressures in ASJ-2J mice.

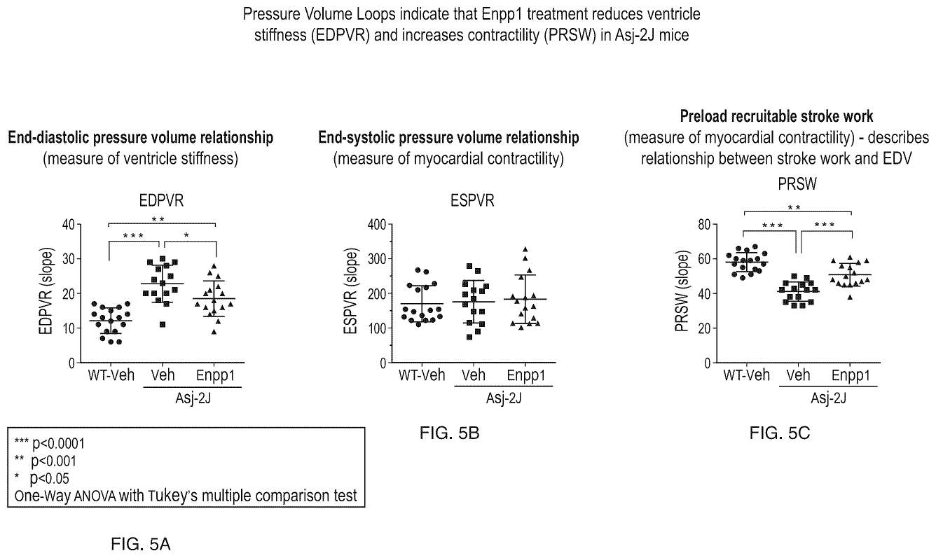

Pressure volume loops were used to assess intact heart function, as well as compliance (stiffness) and contractility of the heart. Pressure volume loops provide simultaneous real-time measurement of both LV pressure and LV volume during a complete cardiac cycle. “ESPVR” describes the maximum pressure that can develop in ventricle at any given LV volume (e.g., it is a measure of myocardial contractility). “EDPVR” describes the passive filling curve for the ventricle and is a measure of passive chamber stiffness (e.g., it is a measure of ventricular stiffness). “PRSW” describes the relationship between stroke work and EDV (e.g., it is a measure of myocardial contractility). As shown in FIGS. 5A-5C the pressure volume loops indicate that ENPP1 treatment reduces ventricle stiffness (EDPVR) and increases contractility (PRSW) in ASJ-2J mice.

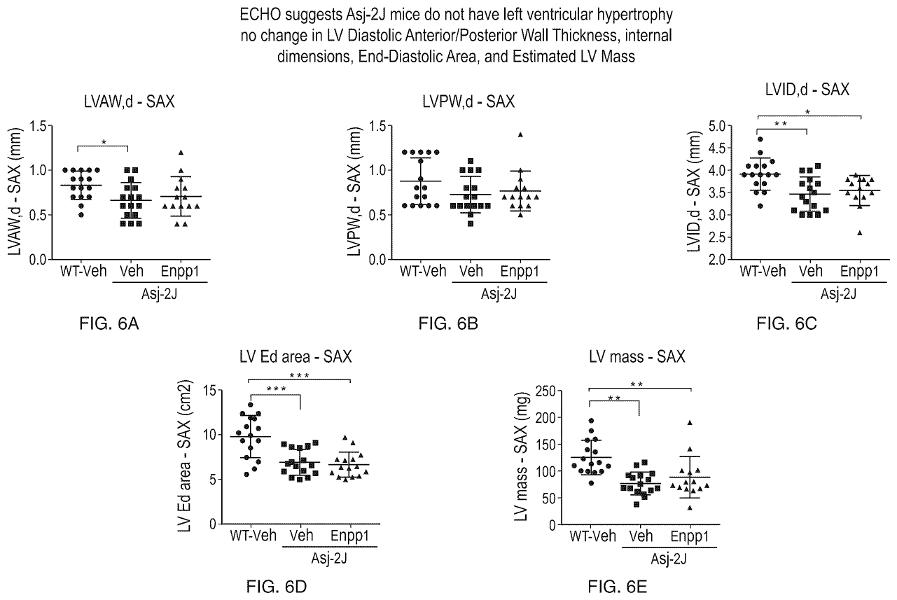

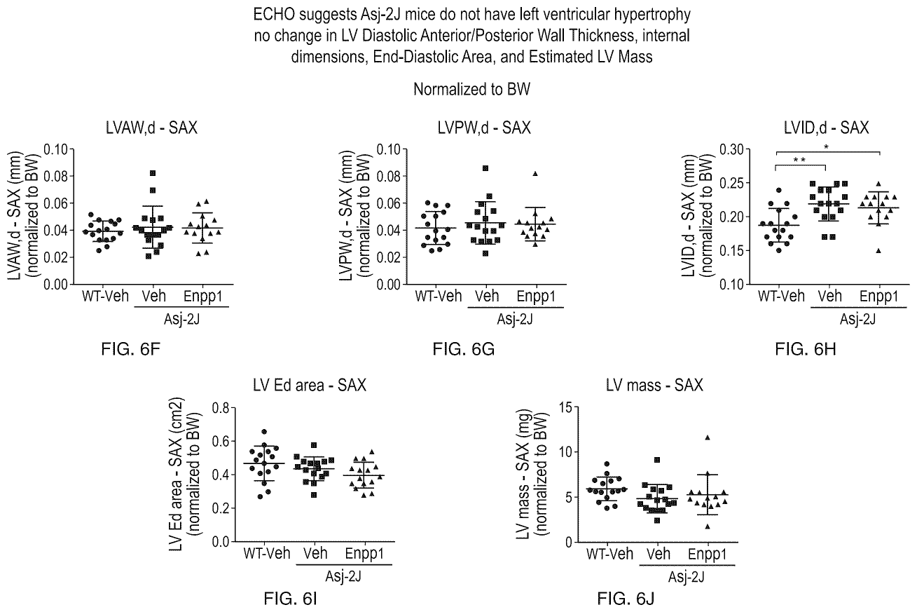

An echocardiogram (“ECHO”) is a test that uses high frequency sound waves (ultrasound) to create pictures of the heart's chambers, valves, walls and the blood vessels (aorta, arteries, veins) attached to the heart. As shown in FIGS. 6A-6J, the results of the echocardiogram suggest that Asj-2J mice do not have left ventricular hypertrophy, as there was no change in LV Diastolic Anterior/Posterior Wall Thickness, internal dimensions, End-Diastolic Area, and Estimated LV Mass.

In summary, Enpp1 treatment improved the elevated blood pressure (SBP, DBP, MBP) and left ventricular pressure observed in Asj-2J mice. HR was unaffected. The pressure volume loop data suggests that Asj-2J have increased ventricle stiffness (EDPVR) and reduced contractility (PRSW), both of which were rescued by Enpp1 treatment. ECHO analysis of Asj-2J mice (normalized to BW) indicated no change in cardiac dimensions and area, suggesting no indication of left ventricular hypertrophy. Overall, there a significant improvement in cardiovascular function was observed with Enpp1 treatment, despite no reduction in calcification This suggests that improvements in cardiovascular function could be due to calcification-independent effects, such as adenosine signaling.

### Example 2: Evaluation of Feasibility of Hemodynamic Procedure in Young Asj-2J Mice

It is hypothesized that if Enpp1 can improve blood pressure in 4 week old Asj-2J mice (before elevated calcification detected), Enpp1-induced improvements in blood pressure are are independent of improvements in calcification, thus suggesting a possible role of Enpp1 on adenosine signaling and myointimal proliferation. Accordingly, a pilot study is conducted to evaluate the feasibility of hemodynamic procedure in young WT and Asj-2J mice.

The primary objectives are to determine the feasibility of catheterization for the hemodynamic procedure in 4 week WT and Asj-2J mice, determine if blood pressure is elevated in 4 week old Asj-2J mice similar to 8 week old mice, and to determine if isoproterenol (b-adrenergic agonist, which increases heart rate and myocardial contractility) or phenylephrine (a-adrenergic agonist; a vasopressor that increases blood pressure, but does not affect contractility and output of cardiac muscle) challenges uncover any additional differences in responsiveness in the WT and Asj-2J mice. Primary outcome measures include: terminal hemodynamics at baseline and after isoproterenol/phenylephrine challenges (e.g., SAP, DAP, MAP, PP, HR, LVP readouts). Secondary outcome measures include ECHO as a back-up, if hemodynamics are not feasible (cardiac dimensions, HR, FS).

SUMMARY OF SEQUENCE LISTING
amino acid sequence of wild-type NPP1 protein
SEQ ID NO: 1
MERDGCAGGGSRGGEGGRAPREGPAGNGRDRGRSHAAEAPGDPQAAASLLAPMDVGE
EPLEKAARARTAKDPNTYKVLSLVLSVCVLTTILGCIFGLKPSCAKEVKSCKGRCFERTF
GNCRCDAACVELGNCCLDYQETCIEPEHIWTCNKFRCGEKRLTRSLCACSDDCKDKGD
CCINYSSVCQGEKSWVEEPCESINEPQCPAGFETPPTLLFSLDGFRAEYLHTWGGLLPVI
SKLKKCGTYTKNMRPVYPTKTFPNHYSIVTGLYPESHGIIDNKMYDPKMNASFSLKSKE
KFNPEWYKGEPIWVTAKYQGLKSGTFFWPGSDVEINGIFPDIYKMYNGSVPFEERILAVL
QWLQLPKDERPHFYTLYLEEPDSSGHSYGPVSSEVIKALQRVDGMVGMLMDGLKELNL
HRCLNLILISDHGMEQGSCKKYIYLNKYLGDVKNIKVIYGPAARLRPSDVPDKYYSFNYE
GIARNLSCREPNQHFKPYLKHFLPKRLHFAKSDRIEPLTFYLDPQWQLALNPSERKYCGS
GFHGSDNVFSNMQALFVGYGPGFKHGIEADTFENIEVYNLMCDLLNLTPAPNNGTHGSL
NHLLKNPVYTPKHPKEVHPLVQCPFTRNPRDNLGCSCNPSILPIEDFQTQFNLTVAEEKIIK
HETLPYGRPRVLQKENTICLLSQHQFMSGYSQDILMPLWTSYTVDRNDSFSTEDFSNCLY
QDFRIPLSPVHKCSFYKNNTKVSYGFLSPPQLNKNSSGIYSEALLTTNIVPMYQSFQVIWR
YFHDTLLRKYAEERNGVNVVSGPVFDFDYDGRCDSLENLRQKRRVIRNQEILIPTHFFIVL
TSCKDTSQTPLHCENLDTLAFILPHRTDNSESCVHGKHDSSWVEELLMLHRARITDVEHI
TGLSFYQQRKEPVSDILKLKTHLPTFSQED
amino acid sequence of sNPP1 that contains cysteine-rich region,
catalytic region and c-terminal region
SEQ ID NO: 2
PSCAKEVKSCKGRCFERTFGNCRCDAACVELGNCCLDYQETCIEPEHIWTCNKFRCGEK
RLTRSLCACSDDCKDKGDCCINYSSVCQGEKSWVEEPCESINEPQCPAGFETPPTLLFSL
DGFRAEYLHTWGGLLPVISKLKKCGTYTKNMRPVYPTKTFPNHYSIVTGLYPESHGIIDN
KMYDPKMNASFSLKSKEKFNPEWYKGEPIWVTAKYQGLKSGTFFWPGSDVEINGIFPDI
YKMYNGSVPFEERILAVLQWLQLPKDERPHFYTLYLEEPDSSGHSYGPVSSEVIKALQR
VDGMVGMLMDGLKELNLHRCLNLILISDHGMEQGSCKKYIYLNKYLGDVKNIKVIYGP
AARLRPSDVPDKYYSFNYEGIARNLSCREPNQHFKPYLKHFLPKRLHFAKSDRIEPLTFY
LDPQWQLALNPSERKYCGSGFHGSDNVFSNMQALFVGYGPGFKHGIEADTFENIEVYN
LMCDLLNLTPAPNNGTHGSLNHLLKNPVYTPKHPKEVHPLVQCPFTRNPRDNLGCSCNP
SILPIEDFQTQFNLTVAEEKIIKHETLPYGRPRVLQKENTICLLSQHQFMSGYSQDILMPL
WTSYTVDRNDSFSTEDFSNCLYQDFRIPLSPVHKCSFYKNNTKVSYGFLSPPQLNKNSSG
IYSEALLTTNIVPMYQSFQVIWRYFHDTLLRKYAEERNGVNVVSGPVFDFDYDGRCDSL
ENLRQKRRVIRNQEILIPTHFFIVLTSCKDTSQTPLHCENLDTLAFILPHRTDNSESCVHGK
HDSSWVEELLMLHRARITDVEHITGLSFYQQRKEPVSDILKLKTHLPTFSQED
amino acid sequence of sNPP1-Fc fusion protein
SEQ ID NO: 3
PSCAKEVKSCKGRCFERTFGNCRCDAACVELGNCCLDYQETCIEPEHIWTCNKFRCGEK
RLTRSLCACSDDCKDKGDCCINYSSVCQGEKSWVEEPCESINEPQCPAGFETPPTLLFSL
DGFRAEYLHTWGGLLPVISKLKKCGTYTKNMRPVYPTKTFPNHYSIVTGLYPESHGIIDN
KMYDPKMNASFSLKSKEKFNPEWYKGEPIWVTAKYQGLKSGTFFWPGSDVEINGIFPDI
YKMYNGSVPFEERILAVLQWLQLPKDERPHFYTLYLEEPDSSGHSYGPVSSEVIKALQR
VDGMVGMLMDGLKELNLHRCLNLILISDHGMEQGSCKKYIYLNKYLGDVKNIKVIYGP
AARLRPSDVPDKYYSFNYEGIARNLSCREPNQHFKPYLKHFLPKRLHFAKSDRIEPLTFY
LDPQWQLALNPSERKYCGSGFHGSDNVFSNMQALFVGYGPGFKHGIEADTFENIEVYN
LMCDLLNLTPAPNNGTHGSLNHLLKNPVYTPKHPKEVHPLVQCPFTRNPRDNLGCSCNP
SILPIEDFQTQFNLTVAEEKIIKHETLPYGRPRVLQKENTICLLSQHQFMSGYSQDILMPL
WTSYTVDRNDSFSTEDFSNCLYQDFRIPLSPVHKCSFYKNNTKVSYGFLSPPQLNKNSSG
IYSEALLTTNIVPMYQSFQVIWRYFHDTLLRKYAEERNGVNVVSGPVFDFDYDGRCDSL
ENLRQKRRVIRNQEILIPTHFFIVLTSCKDTSQTPLHCENLDTLAFILPHRTDNSESCVHGK
HDSSWVEELLMLHRARITDVEHITGLSFYQQRKEPVSDILKLKTHLPTFSQEDPKSCDKT
HTCPPCPAPEAAGAPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPAPIEKTISKAK
GQPREPQVYTLPPSREEMTKNQVSLTCLVKGFYPSDIAVEWESNGQPENNYKTTPPVLD
SDGSFFLYSKLTVDKSRWQQGNVFSCSVMHEALHNHYTQKSLSLSPGK
amino acid sequence of sNPP1-Fc-D10
SEQ ID NO: 4
PSCAKEVKSCKGRCFERTFGNCRCDAACVELGNCCLDYQETCIEPEHIWTCNKFRCGEK
RLTRSLCACSDDCKDKGDCCINYSSVCQGEKSWVEEPCESINEPQCPAGFETPPTLLFSL
DGFRAEYLHTWGGLLPVISKLKKCGTYTKNMRPVYPTKTFPNHYSIVTGLYPESHGIIDN
KMYDPKMNASFSLKSKEKFNPEWYKGEPIWVTAKYQGLKSGTFFWPGSDVEINGIFPDI
YKMYNGSVPFEERILAVLQWLQLPKDERPHFYTLYLEEPDSSGHSYGPVSSEVIKALQR
VDGMVGMLMDGLKELNLHRCLNLILISDHGMEQGSCKKYIYLNKYLGDVKNIKVIYGP
AARLRPSDVPDKYYSFNYEGIARNLSCREPNQHFKPYLKHFLPKRLHFAKSDRIEPLTFY
LDPQWQLALNPSERKYCGSGFHGSDNVFSNMQALFVGYGPGFKHGIEADTFENIEVYN
LMCDLLNLTPAPNNGTHGSLNHLLKNPVYTPKHPKEVHPLVQCPFTRNPRDNLGCSCNP
SILPIEDFQTQFNLTVAEEKIIKHETLPYGRPRVLQKENTICLLSQHQFMSGYSQDILMPL
WTSYTVDRNDSFSTEDFSNCLYQDFRIPLSPVHKCSFYKNNTKVSYGFLSPPQLNKNSSG
IYSEALLTTNIVPMYQSFQVIWRYFHDTLLRKYAEERNGVNVVSGPVFDFDYDGRCDSL
ENLRQKRRVIRNQEILIPTHFFIVLTSCKDTSQTPLHCENLDTLAFILPHRTDNSESCVHGK
HDSSWVEELLMLHRARITDVEHITGLSFYQQRKEPVSDILKLKTHLPTFSQEDPKSCDKT
HTCPPCPAPEAAGAPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPAPIEKTISKAK
GQPREPQVYTLPPSREEMTKNQVSLTCLVKGFYPSDIAVEWESNGQPENNYKTTPPVLD
SDGSFFLYSKLTVDKSRWQQGNVFSCSVMHEALHNHYTQKSLSLSPGK
DDDDDDDDDD
amino acid sequences of soluble NPP1 containing amino acids from
107 to 925 of SEQ ID NO: 1
SEQ ID NO: 5
SCKGRCFERTFGNCRCDAACVELGNCCLDYQETCIEPEHIWTCNKFRCGEKRLTRSLCA
CSDDCKDKGDCCINYSSVCQGEKSWVEEPCESINEPQCPAGFETPPTLLFSLDGFRAEYL
HTWGGLLPVISKLKKCGTYTKNMRPVYPTKTFPNHYSIVTGLYPESHGIIDNKMYDPKM
NASFSLKSKEKFNPEWYKGEPIWVTAKYQGLKSGTFFWPGSDVEINGIFPDIYKMYNGS
VPFEERILAVLQWLQLPKDERPHFYTLYLEEPDSSGHSYGPVSSEVIKALQRVDGMVGML
MDGLKELNLHRCLNLILISDHGMEQGSCKKYIYLNKYLGDVKNIKVIYGPAARLRPSDV
PDKYYSFNYEGIARNLSCREPNQHFKPYLKHFLPKRLHFAKSDRIEPLTFYLDPQWQLAL
NPSERKYCGSGFHGSDNVFSNMQALFVGYGPGFKHGIEADTFENIEVYNLMCDLLNLTP
APNNGTHGSLNHLLKNPVYTPKHPKEVHPLVQCPFTRNPRDNLGCSCNPSILPIEDFQTQ
FNLTVAEEKIIKHETLPYGRPRVLQKENTICLLSQHQFMSGYSQDILMPLWTSYTVDRND
SFSTEDFSNCLYQDFRIPLSPVHKCSFYKNNTKVSYGFLSPPQLNKNSSGIYSEALLTTNIV
PMYQSFQVIWRYFHDTLLRKYAEERNGVNVVSGPVFDFDYDGRCDSLENLRQKRRVIR
NQEILIPTHFFIVLTSCKDTSQTPLHCENLDTLAFILPHRTDNSESCVHGKHDSSWVEELL
MLHRARITDVEHITGLSFYQQRKEPVSDILKLKTHLPTFSQED
amino acid sequence of soluble NPP1 containing amino acids from
187 to 925 of SEQ ID NO: 1
SEQ ID NO: 6
EKSWVEEPCESINEPQCPAGFETPPTLLFSLDGFRAEYLHTWGGLLPVISKLKKCGTYTK
NMRPVYPTKTFPNHYSIVTGLYPESHGIIDNKMYDPKMNASFSLKSKEKFNPEWYKGEPI
WVTAKYQGLKSGTFFWPGSDVEINGIFPDIYKMYNGSVPFEERILAVLQWLQLPKDERP
HFYTLYLEEPDSSGHSYGPVSSEVIKALQRVDGMVGMLMDGLKELNLHRCLNLILISDH
GMEQGSCKKYIYLNKYLGDVKNIKVIYGPAARLRPSDVPDKYYSFNYEGIARNLSCREP
NQHFKPYLKHFLPKRLHFAKSDRIEPLTFYLDPQWQLALNPSERKYCGSGFHGSDNVFS
NMQALFVGYGPGFKHGIEADTFENIEVYNLMCDLLNLTPAPNNGTHGSLNHLLKNPVY
TPKHPKEVHPLVQCPFTRNPRDNLGCSCNPSILPIEDFQTQFNLTVAEEKIIKHETLPYGRP
RVLQKENTICLLSQHQFMSGYSQDILMPLWTSYTVDRNDSFSTEDFSNCLYQDFRIPLSP
VHKCSFYKNNTKVSYGFLSPPQLNKNSSGIYSEALLTTNIVPMYQSFQVIWRYFHDTLLR
KYAEERNGVNVVSGPVFDFDYDGRCDSLENLRQKRRVIRNQEILIPTHFFIVLTSCKDTS
QTPLHCENLDTLAFILPHRTDNSESCVHGKHDSSWVEELLMLHRARITDVEHITGLSFYQ
QRKEPVSDILKLKTHLPTFSQED
amino acid sequence of Fc region of human IgG1 including hinge
region
SEQ ID NO: 7
EPKSCDKTHTCPPCPAPELLGGPSVFLFPPKPKDTLMISRTPEVTCVVVDVSHEDPEVKF
NWYVDGVEVHNAKTKPREEQYNSTYRVVSVLTVLHQDWLNGKEYKCKVSNKALPAPI
EKTISKAKGQPREPQVYTLPPSREEMTKNQVSLTCLVKGFYPSDIAVEWESNGQPENNY
KTTPPVLDSDGSFFLYSKLTVDKSRWQQGNVFSCSVMHEALHNHYTQKSLSLSPGK
amino acid sequence of Fc of human IgG1 including partial hinge
region
SEQ ID NO: 8
DKTHTCPPCPAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKTIS
KAKGQPREPQVYTLPPSREEMTKNQVSLTCLVKGFYPSDIAVEWESNGQPENNYKTTPP
VLDSDGSFFLYSKLTVDKSRWQQGNVFSCSVMHEALHNHYTQKSLSLSPGK
amino acid sequence of NPP1-Fc fusion protein [(107-925)-Fc]
SEQ ID NO: 9
SCKGRCFERTFGNCRCDAACVELGNCCLDYQETCIEPEHIWTCNKFRCGEKRLTRSLCA
CSDDCKDKGDCCINYSSVCQGEKSWVEEPCESINEPQCPAGFETPPTLLFSLDGFRAEYL
HTWGGLLPVISKLKKCGTYTKNMRPVYPTKTFPNHYSIVTGLYPESHGIIDNKMYDPKM
NASFSLKSKEKFNPEWYKGEPIWVTAKYQGLKSGTFFWPGSDVEINGIFPDIYKMYNGS
VPFEERILAVLQWLQLPKDERPHFYTLYLEEPDSSGHSYGPVSSEVIKALQRVDGMVGML
MDGLKELNLHRCLNLILISDHGMEQGSCKKYIYLNKYLGDVKNIKVIYGPAARLRPSDV
PDKYYSFNYEGIARNLSCREPNQHFKPYLKHFLPKRLHFAKSDRIEPLTFYLDPQWQLAL
NPSERKYCGSGFHGSDNVFSNMQALFVGYGPGFKHGIEADTFENIEVYNLMCDLLNLTP
APNNGTHGSLNHLLKNPVYTPKHPKEVHPLVQCPFTRNPRDNLGCSCNPSILPIEDFQTQ
FNLTVAEEKIIKHETLPYGRPRVLQKENTICLLSQHQFMSGYSQDILMPLWTSYTVDRND
SFSTEDFSNCLYQDFRIPLSPVHKCSFYKNNTKVSYGFLSPPQLNKNSSGIYSEALLTTNIV
PMYQSFQVIWRYFHDTLLRKYAEERNGVNVVSGPVFDFDYDGRCDSLENLRQKRRVIR
NQEILIPTHFFIVLTSCKDTSQTPLHCENLDTLAFILPHRTDNSESCVHGKHDSSWVEELL
MLHRARITDVEHITGLSFYQQRKEPVSDILKLKTHLPTFSQEDEPKSCDKTHTCPPCPAPE
LLGGPSVFLFPPKPKDTLMISRTPEVTCVVVDVSHEDPEVKFNWYVDGVEVHNAKTKPR
EEQYNSTYRVVSVLTVLHQDWLNGKEYKCKVSNKALPAPIEKTISKAKGQPREPQVYTL
PPSREEMTKNQVSLTCLVKGFYPSDIAVEWESNGQPENNYKTTPPVLDSDGSFFLYSKLT
VDKSRWQQGNVFSCSVMHEALHNHYTQKSLSLSPGK
amino acid sequence of NPP1-Fc fusion protein [(107-925)-partial
hinge Fc]
SEQ ID NO: 10
SCKGRCFERTFGNCRCDAACVELGNCCLDYQETCIEPEHIWTCNKFRCGEKRLTRSLCA
CSDDCKDKGDCCINYSSVCQGEKSWVEEPCESINEPQCPAGFETPPTLLFSLDGFRAEYL
HTWGGLLPVISKLKKCGTYTKNMRPVYPTKTFPNHYSIVTGLYPESHGIIDNKMYDPKM
NASFSLKSKEKFNPEWYKGEPIWVTAKYQGLKSGTFFWPGSDVEINGIFPDIYKMYNGS
VPFEERILAVLQWLQLPKDERPHFYTLYLEEPDSSGHSYGPVSSEVIKALQRVDGMVGML
MDGLKELNLHRCLNLILISDHGMEQGSCKKYIYLNKYLGDVKNIKVIYGPAARLRPSDV
PDKYYSFNYEGIARNLSCREPNQHFKPYLKHFLPKRLHFAKSDRIEPLTFYLDPQWQLAL
NPSERKYCGSGFHGSDNVFSNMQALFVGYGPGFKHGIEADTFENIEVYNLMCDLLNLTP
APNNGTHGSLNHLLKNPVYTPKHPKEVHPLVQCPFTRNPRDNLGCSCNPSILPIEDFQTQ
FNLTVAEEKIIKHETLPYGRPRVLQKENTICLLSQHQFMSGYSQDILMPLWTSYTVDRND
SFSTEDFSNCLYQDFRIPLSPVHKCSFYKNNTKVSYGFLSPPQLNKNSSGIYSEALLTTNIV
PMYQSFQVIWRYFHDTLLRKYAEERNGVNVVSGPVFDFDYDGRCDSLENLRQKRRVIR
NQEILIPTHFFIVLTSCKDTSQTPLHCENLDTLAFILPHRTDNSESCVHGKHDSSWVEELL
MLHRARITDVEHITGLSFYQQRKEPVSDILKLKTHLPTFSQEDDKTHTCPPCPAPELLGGP
SVFLFPPKPKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREPQVYTLPPSR
EEMTKNQVSLTCLVKGFYPSDIAVEWESNGQPENNYKTTPPVLDSDGSFFLYSKLTVDK
SRWQQGNVFSCSVMHEALHNHYTQKSLSLSPGK
amino acid sequence of NPP1-Fc fusion protein [(187-925)-Fc]
SEQ ID NO: 11
EKSWVEEPCESINEPQCPAGFETPPTLLFSLDGFRAEYLHTWGGLLPVISKLKKCGTYTK
NMRPVYPTKTFPNHYSIVTGLYPESHGIIDNKMYDPKMNASFSLKSKEKFNPEWYKGEPI
WVTAKYQGLKSGTFFWPGSDVEINGIFPDIYKMYNGSVPFEERILAVLQWLQLPKDERP
HFYTLYLEEPDSSGHSYGPVSSEVIKALQRVDGMVGMLMDGLKELNLHRCLNLILISDH
GMEQGSCKKYIYLNKYLGDVKNIKVIYGPAARLRPSDVPDKYYSFNYEGIARNLSCREP
NQHFKPYLKHFLPKRLHFAKSDRIEPLTFYLDPQWQLALNPSERKYCGSGFHGSDNVFS
NMQALFVGYGPGFKHGIEADTFENIEVYNLMCDLLNLTPAPNNGTHGSLNHLLKNPVY
TPKHPKEVHPLVQCPFTRNPRDNLGCSCNPSILPIEDFQTQFNLTVAEEKIIKHETLPYGRP
RVLQKENTICLLSQHQFMSGYSQDILMPLWTSYTVDRNDSFSTEDFSNCLYQDFRIPLSP
VHKCSFYKNNTKVSYGFLSPPQLNKNSSGIYSEALLTTNIVPMYQSFQVIWRYFHDTLLR
KYAEERNGVNVVSGPVFDFDYDGRCDSLENLRQKRRVIRNQEILIPTHFFIVLTSCKDTS
QTPLHCENLDTLAFILPHRTDNSESCVHGKHDSSWVEELLMLHRARITDVEHITGLSFYQ
QRKEPVSDILKLKTHLPTFSQEDEPKSCDKTHTCPPCPAPELLGGPSVFLFPPKPKDTLMI
SRTPEVTCVVVDVSHEDPEVKFNWYVDGVEVHNAKTKPREEQYNSTYRVVSVLTVLH
QDWLNGKEYKCKVSNKALPAPIEKTISKAKGQPREPQVYTLPPSREEMTKNQVSLTCLV
KGFYPSDIAVEWESNGQPENNYKTTPPVLDSDGSFFLYSKLTVDKSRWQQGNVFSCSV
MHEALHNHYTQKSLSLSPGK
amino acid sequence of NPP1-Fc fusion protein [(187-925)-partial
hinge Fc]
SEQ ID NO: 12
EKSWVEEPCESINEPQCPAGFETPPTLLFSLDGFRAEYLHTWGGLLPVISKLKKCGTYTK
NMRPVYPTKTFPNHYSIVTGLYPESHGIIDNKMYDPKMNASFSLKSKEKFNPEWYKGEPI
WVTAKYQGLKSGTFFWPGSDVEINGIFPDIYKMYNGSVPFEERILAVLQWLQLPKDERP
HFYTLYLEEPDSSGHSYGPVSSEVIKALQRVDGMVGMLMDGLKELNLHRCLNLILISDH
GMEQGSCKKYIYLNKYLGDVKNIKVIYGPAARLRPSDVPDKYYSFNYEGIARNLSCREP
NQHFKPYLKHFLPKRLHFAKSDRIEPLTFYLDPQWQLALNPSERKYCGSGFHGSDNVFS
NMQALFVGYGPGFKHGIEADTFENIEVYNLMCDLLNLTPAPNNGTHGSLNHLLKNPVY
TPKHPKEVHPLVQCPFTRNPRDNLGCSCNPSILPIEDFQTQFNLTVAEEKIIKHETLPYGRP
RVLQKENTICLLSQHQFMSGYSQDILMPLWTSYTVDRNDSFSTEDFSNCLYQDFRIPLSP
VHKCSFYKNNTKVSYGFLSPPQLNKNSSGIYSEALLTTNIVPMYQSFQVIWRYFHDTLLR
KYAEERNGVNVVSGPVFDFDYDGRCDSLENLRQKRRVIRNQEILIPTHFFIVLTSCKDTS
QTPLHCENLDTLAFILPHRTDNSESCVHGKHDSSWVEELLMLHRARITDVEHITGLSFYQ
QRKEPVSDILKLKTHLPTFSQEDDKTHTCPPCPAPELLGGPSVFLFPPKPKDTLMISRTPE
VTCVVVDVSHEDPEVKFNWYVDGVEVHNAKTKPREEQYNSTYRVVSVLTVLHQDWL
NGKEYKCKVSNKALPAPIEKTISKAKGQPREPQVYTLPPSREEMTKNQVSLTCLVKGFY
PSDIAVEWESNGQPENNYKTTPPVLDSDGSFFLYSKLTVDKSRWQQGNVFSCSVMHEA
LHNHYTQKSLSLSPGK
human IgG1 hinge region
SEQ ID NO: 13
EPKSCDKTHTCPPCP
portion of human IgG1 hinge region
SEQ ID NO: 14
DKTHTCPPCP
portion of human IgG1 hinge region
SEQ ID NO: 15
PKSCDKTHTCPPCP
Linker
SEQ ID NO: 16
(Gly₄Ser)₃
amino acid motif that is start of soluble NPP1 which includes
cysteine rich region
SEQ ID NO: 17
PSCAKE
SEQ ID NO: 18
D10 targeting moiety
synthetic linker
19
(Gly₄Ser)ₙ,

What is claimed is:

1. A method for improving cardiovascular function in a human subject, the method comprising administering to the patient one or more doses of a recombinant human soluble ectonucleotide pyrophosphatase phosphodiesterase (hsNPP1), active fragment or fusion protein thereof, and wherein said subject is not ENPP1 deficient.

2. A method for treating a cardiovascular disease in a human subject, the method comprising administering to the patient one or more doses of a recombinant human soluble ectonucleotide pyrophosphatase phosphodiesterase (hsNPP1), active fragment or fusion protein thereof, and wherein said subject is not ENPP1 deficient.

3. A method for treating a human subject having hypertension, the method comprising administering to the subject one or more doses of a recombinant human soluble ectonucleotide pyrophosphatase phosphodiesterase (hsNPP1), active fragment or fusion protein thereof, and wherein said subject is not ENPP1 deficient.

4. A method for reducing hypertension in a human subject, the method comprising administering to the subject one or more doses of a recombinant human soluble ectonucleotide pyrophosphatase phosphodiesterase (hsNPP1), active fragment or fusion protein thereof, and wherein said subject is not ENPP1 deficient.

5. The method of claim 2 or 3, wherein the hsNPP1 is a fusion protein.

6. The method of claim 5, wherein the fusion protein comprises an Fc region of an immunoglobulin.

7. The method of claim 5, wherein the fusion protein comprises a targeting moiety.

8. The method of claim 7, wherein said targeting moiety comprises at least eight consecutive aspartic acid or glutamic acid residues (SEQ ID NOS 20 and 21, respectively).

9. The method of claim 5, wherein the fusion protein comprises SEQ ID NO:3, SEQ ID NO:4, SEQ ID NO:9, SEQ ID NO:10, SEQ ID NO:11 or SEQ ID NO:12.

10. The method of any one of the preceding claims, wherein the one or more doses contains about 1.0 mg/kg to about 20.0 mg/kg NPP1.

11. The method of claim 2 or 3, wherein the one or more doses contains about 1.0 mg/kg to about 5.0 mg/kg NPP1.

12. The method of claim 2 or 3, wherein the one or more doses is about 0.2 mg/kg, about 0.5 mg/kg, about 1 mg/kg, about 5.0 mg/kg, about 6.0 mg/kg, about 10 mg/kg, about 15 mg/kg, or about 20 mg/kg.

13. The method of claim 2 or 3, wherein two or more doses of NPP1 are administered at least 3 days, 1 week, 2 weeks or 1 month apart.

14. The method of claim 2 or 3, wherein the administration is weekly, bi-weekly, or monthly.

15. The method of claim 2 or 3, wherein the administration is intravenous, subcutaneous, or intraperitoneal.

16. The method of claim 2 or 3, wherein an additional therapeutic agent is administered to the subject.

17. The method of claim 2 or 3, wherein the one or more doses is 0.2 mg/kg (+/−20%), 0.5mg/kg (+/−20%), 1 mg/kg (+/−20%), 2.0 mg/kg (+/−20%), 5.0 mg/kg (+/−20%), 6.0 mg/kg (+/−20%), 10 mg/kg (+/−20%), 15 mg/kg (+/−20%), or 20 mg/kg (+/−20%).

18. The method of claim 3 or 4, wherein two or more doses of NPP1 are administered at least 3 days, 1 week, 2 weeks or 1 month apart.

19. The method of claim 1, wherein the improvement is a reduction in elevated blood pressure, normalization of blood pressure, a reduction in left ventricular end-diastolic pressure (EDP), a reduction in left ventricular end-systolic pressure (ESP), a reduction in ventricle stiffness, and/or an increase in contractility.

20. The method of claim 17, wherein the reduction in elevated blood pressure is a reduction in blood pressure higher than about 140 over 90 millimeters of mercury (mmHg) to about 120 over 80 mm of mercury (mmHg).
